(19) United States
(12) Patent Application Publication
(10) Pub. No.: US 2016/0081602 A1
(43) Pub. Date: Mar. 24, 2016

(54) METHODS AND SYSTEMS FOR PROVIDING POWER TO LIGHT SOURCES OF A PHYSIOLOGICAL MONITOR

(71) Applicant: Covidien LP, Mansfield, MA (US)

(72) Inventors: Daniel Lisogurki, Boulder, CO (US); Christopher Meehan, Golden, CO (US); William Kit Dean, Castle Pines, CO (US)

(21) Appl. No.: 14/857,710

(22) Filed: Sep. 17, 2015

Related U.S. Application Data
(60) Provisional application No. 62/052,363, filed on Sep. 18, 2014.

(51) Int. Cl. A61B 5/1455

(52) U.S. Cl. CPC A61B 5/14551

(57) ABSTRACT

Systems and methods for providing power to a light source of a physiological system and for generating a desired signal. The physiological system may comprise a light drive circuit that may provide a digital signal to the light source of the physiological system. The digital signal may comprise a plurality of pulses. The plurality of pulses may include one or more features that may be adjustable to vary the power provided to the light source. For example, the pulses may have a varying width. The light drive circuit may be configured to provide power to the light source during each pulse of the plurality of pulses. The system may further comprise a front end circuit configured to receive the light generated by the light source in response to the plurality of pulses, where the light is attenuated by a body tissue of a patient. The front end circuit may comprise a response time. The at least one period of the plurality of pulses may be substantially shorter than the response time of the front end channel circuit.

The present disclosure relates to generating and processing signals in a physiological monitor, and more particularly relates to techniques for providing power to one or more light sources of the physiological monitor in order to generate a desired signal.

### SUMMARY

The present disclosure is directed towards providing power to a light source of a physiological monitoring system, such as a medical device. Methods and systems are provided for providing power to the light source of the physiological monitor. In some embodiments, the power is provided by a system comprising a light drive circuit and a front end channel circuit. The light drive circuit of the system may be configured to provide a digital signal that provides power to a light source of the system. In some embodiments, the digital signal may comprise a plurality of pulses where the pulses comprise a feature that is adjustable to vary the power provided to the light source. The plurality of pulses may also comprise one or more periods. The front end channel circuit of the system may be configured to receive light generated by the light source of the system in response to the plurality of pulses, after the light has been attenuated by body tissue. The front end channel circuit may comprise a response time (e.g., channel bandwidth or frequency response time), such that the one or more periods of the plurality of pulses are substantially shorter than the response time of the front end channel circuit.

In some embodiments, the power is provided by a system comprising a light drive, a light source and a front end channel. The light drive may be configured to provide a digital control signal that provides power to a light source. The digital signal may comprise a plurality of pulses. The light drive may be further configured to provide power to the light source during each pulse of the plurality of pulses. The front end channel may be configured to receive pulses of light in response to the plurality of pulses. The pulses of light may be attenuated by a body tissue during each pulse of the plurality of pulses. The front end channel may further be configured to integrate or filter (such as with a low pass or band pass filter) the plurality of pulses of the received signal to generate a desired analog pulse.

In some embodiments, a method is disclosed for providing power to a light source of a physiological monitoring system. The method comprises providing power to a light source according to a digital signal. In some embodiments, the digital signal may comprise a plurality of pulses where the pulses comprise a feature that is adjustable to vary the power provided to the light source. The plurality of pulses may also comprise one or more periods. The method further comprises receiving, by a front end channel, light generated by the one or more light source in response to the plurality of pulses after the light was attenuated by body tissue. The front end channel may comprise a response time, such that the one or periods of the plurality of pulses are substantially shorter than the response time of the front end channel.

In some embodiments, a method is disclosed for providing power to a light source of a physiological monitoring system. The method comprises providing a digital signal that provides power to the light source. In some embodiments, the digital signal may comprise a plurality of pulses such that the power is provided to the light source during each pulse. The method further comprises receiving, by a front end channel, pulses of light generated by the light source in response to the plurality of pulses of the digital signal, wherein the pulses of light are attenuated by a body tissue during each pulse of the plurality of pulses. The method further comprises integrating the plurality of pulses of the received signal to create a desired analog signal.

In some embodiments, the power is provided by a system comprising a light drive circuit and a front end channel circuit. The light drive circuit may be configured to provide a digital signal that provides power to a light source. The digital signal may comprise a plurality of pulses, where the pulses comprise at least two features that are adjustable to vary the power provided to the light source. The light drive circuit may be configured to provide power to the light source during each pulse of the plurality of pulses. The at least two features of the plurality of pulses may comprise at least two of width of the plurality of pulses, amplitude of the plurality of pulses, and pulse frequency of the plurality of pulses. The front end channel circuit may be configured to receive light generated by the light source in response to the plurality of pulses after the light has been attenuated by a body tissue.

In some embodiments, a method is disclosed for providing power to a light source of a physiological monitoring system. The method comprises providing a digital signal to a light source. The digital signal may comprise a plurality of pulses, wherein at least two features of the plurality of pulses are adjustable to vary the power provided to the light source. The power may be provided to the light source during each pulse of the plurality of pulses. The at least two features of the plurality of pulses may comprise at least two of width of the plurality of pulses, amplitude of the plurality of pulses and pulse frequency of the plurality of pulses. The method may further comprise receiving, by a front end channel circuit, light generated by the light source in response to the plurality of pulses after the light has been attenuated by a body tissue.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows a block diagram of an illustrative physiological monitoring system in accordance with some embodiments of the present disclosure;

FIG. 2A shows an illustrative plot of a light drive signal in accordance with some embodiments of the present disclosure;

FIG. 2B shows an illustrative plot of a detector signal that may be generated by a sensor in accordance with some embodiments of the present disclosure;

FIG. 3 is a perspective view of an illustrative physiological monitoring system in accordance with some embodiments of the present disclosure;

FIG. 4A shows an illustrative plot of another light drive signal in accordance with some embodiments of the present disclosure;

FIG. 4B shows an illustrative plot of a detector signal that may be generated by a sensor in accordance with some embodiments of the present disclosure;

FIG. 5A shows an illustrative plot of yet another light drive signal in accordance with some embodiments of the present disclosure;

FIG. 5B shows an illustrative plot of a detector signal that may be generated by a sensor in accordance with some embodiments of the present disclosure;

FIG. 6 shows an illustrative flow diagram including steps for providing power to light sources of a physiological monitor;

FIG. 7 shows a block diagram of an illustrative system for light drive signal generation in accordance with some embodiments of the present disclosure;

FIG. 8 shows a panel of illustrative plots of several light drive signals in accordance with some embodiments of the present disclosure;

FIG. 9 shows a panel of illustrative timing diagrams for generating an exemplary light drive signal in accordance with some embodiments of the present disclosure;

FIG. 10 shows a circuit diagram of an illustrative system for light signal generation in accordance with some embodiments of the present disclosure;

FIG. 11A-C show several illustrative plots of exemplary light drive signals in accordance with some embodiments of the present disclosure; and

FIG. 12 shows a circuit diagram of an illustrative system for adjusting voltage of a power source in accordance with some embodiments of the present disclosure.

### DETAILED DESCRIPTION OF THE FIGURES

The systems and methods described below may be implemented using a photoplethysmography (PPG) device, such as an oximeter. The PPG device may comprise a front end channel configured to receive light generated by a light source, after the light is attenuated by a body tissue of a patient. In some embodiments, the light source may generate pulses and the front-end channel may comprise a response time that is relatively slower than the pulses. The front end channel, and its relatively slower response time, may be used to generate a desired analog signal for further processing.

The PPG device may comprise, for example, a light drive circuit configured to provide a digital signal to one or more light sources. The digital signal may be a digitally modulated (e.g., Pulse Width Modulated) version of a desired analog signal. The digital signal may comprise multiple pulses. The pulses may comprise an adjustable feature (e.g., width), and power may be provided to the light sources during each pulse. The adjustable feature may be used to vary the average power provided to the one or more light sources. The pulses may comprise a pulse frequency. The period corresponding to the pulse frequency may advantageously be significantly shorter than the response time of the front-end channel. For example, the front-end channel may receive light generated according to pulses that are delivered at a rate that is much faster than the response time of the front-end channel. Consequently, the front-end channel may effectively integrate the pulses of the digital signal to generate the desired analog signal.

For purposes of this disclosure, a PPG device may refer to any device that generates or obtains signals based on measured changes in light absorption. In some embodiments, a PPG device may comprise a pulse oximeter configured to illuminate skin or tissue of a subject with light and measure the light after it is attenuated by skin or tissue of the subject. However, other devices that measure changes in light absorption may also be used.

For purposes of this disclosure, a digital signal may refer to any physical or virtual signal that represents a sequence or succession of discrete values. In some embodiments, a digital signal may comprise two or more discrete states. For example, the digital signal may comprise a high state and a low state. In some embodiments, the digital signal may comprise a high state, a low state, and a predefined number of intermediate states. In some embodiments, a digital signal may comprise a series of discrete states connected by rising and falling edges. In other embodiments, a digital signal may be any other kind of sequence of binary or non-binary discrete values. For example, a digital signal may comprise a plurality of modulated square waves. In some embodiments, the digital signal may comprise a pulse-width modulated (PWM) waveform, a pulse-frequency modulated (PFM) waveform, a pulse-density modulated (PDM) waveform, a pulse-amplitude modulated (PAM) waveform, a pulse-position modulated (PPM) waveform, a pulse-code modulated (PCM) waveform, any other suitable modulated waveform, and any combinations thereof.

For purposes of this disclosure, a front end channel may refer to any device or system that is configured to receive, detect, measure or process light generated by any type of a light source. In some embodiments, the front end channel may comprise a front end circuit of an oximetry device. In some embodiments, the front end channel may comprise a light detector connected to a circuit for processing signals generated by the light detector in response to detecting light. The front end channel may comprise any combination of hardware and software necessary to receive, detect, measure or process light signals.

For purposes of this disclosure, a response time may refer to a property of any kind of device or a system that represents the amount of time needed for the device or system to react to an input. For example, the response time of an electrical circuit board may be the amount of time need for the circuit board to react to an electrical signal input. In some embodiments, the response time may refer to the time needed for a front end channel to generate an output signal in response to received input signal. In some embodiments, the response time may comprise the settling time of a circuit board. In some embodiments, the response time may refer to a step response time or settling time of a step input of an electronic device. In some embodiment, the response time may refer to maximum bandwidth of device or a system.

For purposes of this disclosure, an analog pulse may refer to any portion of an analog signal. In some embodiments, an analog pulse may be a single period of an analog signal that comprises a frequency. For example, if an analog signal comprises a sinusoid wave with a certain frequency an analog pulse may comprise a portion of the analog signal over a period defined by the frequency of the analog signal.

As mentioned above, the techniques described herein may be implemented in a PPG device such as an oximeter. An oximeter is a medical device that may determine the oxygen saturation of an analyzed tissue. One common type of oximeter is a pulse oximeter, which may non-invasively measure the oxygen saturation of a patient's blood (as opposed to measuring oxygen saturation invasively by analyzing a blood sample taken from the patient). Pulse oximeters may be included in patient monitoring systems that measure and display various blood flow characteristics including, but not limited to, the blood oxygen saturation (e.g., arterial, venous, or both). Such patient monitoring systems may also measure and display additional or alternative physiological parameters such as pulse rate, respiration rate, respiration effort, blood pressure, hemoglobin concentration (e.g., oxygenated, deoxygenated, and/or total), any other suitable physiological parameters, or any combination thereof. Exemplary embodiments of determining respiration rate are disclosed in Addison et al. U.S. Patent Publication No. 2011/0071406, published Mar. 24, 2011. Exemplary embodiments of determining respiration effort are disclosed in Addison et al. U.S. Patent Publication No. 2011/0004081, published Jan. 6, 2011. Exemplary embodiments of determining blood pressure are disclosed in Addison et al. U.S. Patent Publication No. 2011/0028854, published Feb. 3, 2011.

Pulse oximetry may be implemented using a photoplethysmograph. Pulse oximeters and other photoplethysmograph devices may also be used to determine other physiological parameter and information as disclosed in: J. Allen, “Photoplethysmography and its application in clinical physiological measurement,” Physiol. Meas., vol. 28, pp. R1-R39, March 2007; W. B. Murray and P. A. Foster, “The peripheral pulse wave: information overlooked,” J. Clin. Monit., vol. 12, pp. 365-377, September 1996; A. Reisner, et al., “Utility of the photoplethysmogram in circulatory monitoring,” Anesthesiology, 108(5):950-958, May 2008; and K. H. Shelley, “Photoplethysmography: beyond the calculation of arterial oxygen saturation and heart rate,” Anesth. Analg., vol. 105, pp. S31-S36, December 2007.

An oximeter may include a light sensor that is placed at a site on a patient, typically a fingertip, toe, forehead or earlobe, or in the case of a neonate, across a foot or hand. The oximeter may use a light source to pass light through blood perfused tissue and photoelectrically sense the absorption of the light in the tissue. Additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, around or in front of the ear, and locations with strong pulsatile arterial flow. Suitable sensors for these locations may include sensors that detect reflected light.

The oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, an inverted signal, etc.) may be referred to as the photoplethysmograph (PPG) signal. In addition, the term “PPG signal,” as used herein, may also refer to an intensity or absorption signal (i.e., representing the amount of light absorbed by or passed through the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some embodiments, the photonic signal interacting with the tissue is of one or more wavelengths that are attenuated by the blood in an amount representative of the blood constituent concentration. Red and infrared (IR) wavelengths may be used because it has been observed that highly oxygenated blood will absorb relatively less red light and more IR light than blood with a lower oxygen saturation. By comparing the intensities of two wavelengths at different points in the pulse cycle, it is possible to estimate the blood oxygen saturation of hemoglobin in arterial blood.

For example, the system may determine blood oxygen saturation using two wavelengths of light and a ratio-of-ratios calculation. The system also may identify pulses and determine pulse amplitude, respiration, blood pressure, other suitable parameters, or any combination thereof, using any suitable calculation techniques. In some embodiments, the system may use information from external sources (e.g., tabulated data, secondary sensor devices) to determine physiological parameters.

In some embodiments, a light drive modulation may be used. For example, a first light source may be turned on for a first drive pulse, followed by an off period, followed by a second light source for a second drive pulse, followed by an off period. The first and second drive pulses may be used to determine physiological parameters. The off periods may be used to detect ambient signal levels, reduce overlap of the light drive pulses, allow time for light sources to stabilize, allow time for detected light signals to stabilize or settle, reduce heating effects, reduce power consumption, for any other suitable reason, or any combination thereof.

It will be understood that the techniques described herein are not limited to pulse oximeters and may be applied to any suitable medical and non-medical devices. For example, the system may determine light drive and other system parameters for use in determining physiological parameters such as regional saturation (rSO₂), respiration rate, respiration effort, continuous non-invasive blood pressure, oxygen saturation pattern detection, fluid responsiveness, cardiac output, heart rate, any other suitable physiological parameter, or any combination thereof.

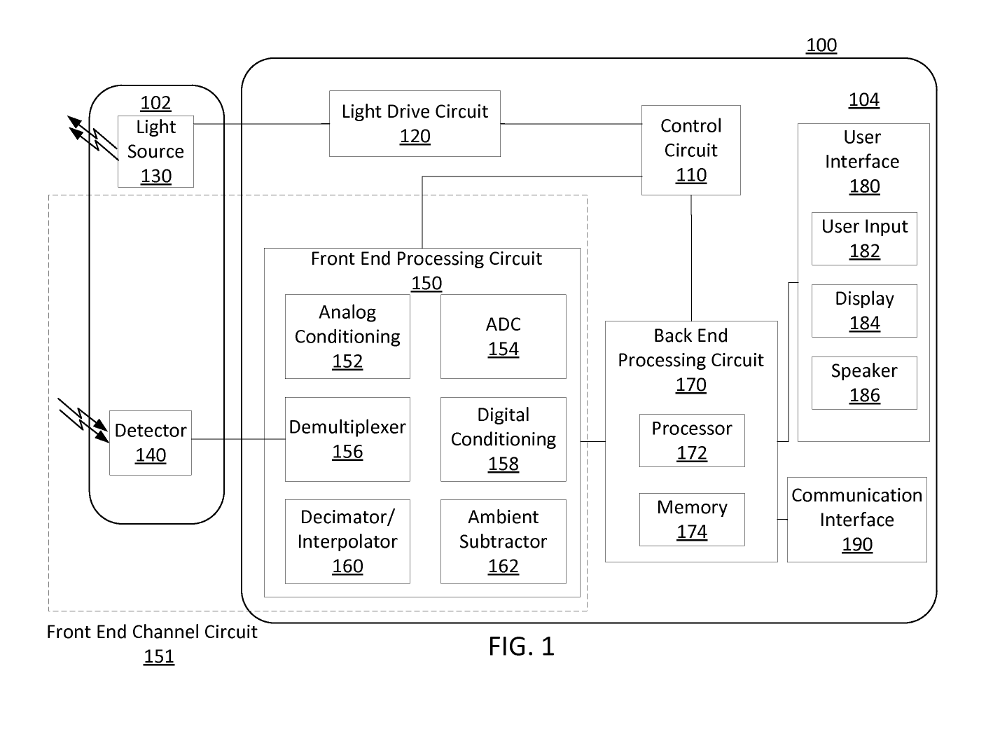

The following description and accompanying FIGS. 1-12 provide additional details and features of some embodiments of the present disclosure.

FIG. 1 shows a block diagram of illustrative physiological monitoring system 100 in accordance with some embodiments of the present disclosure. System 100 may include a sensor 102 and a monitor 104 for generating and processing sensor signals that include physiological information of a subject. In some embodiments, sensor 102 and monitor 104 may be part of an oximeter.

Sensor 102 of physiological monitoring system 100 may include light source 130 and detector 140. Light source 130 may be configured to emit photonic signals having one or more wavelengths of light (e.g., red and IR) into a subject's tissue. For example, light source 130 may include a red light emitting light source and an IR light emitting light source, e.g., red and IR light emitting diodes (LEDs), for emitting light into the tissue of a subject to generate sensor signals that include physiological information. In some embodiments, the red wavelength may be between about 600 nm and about 750 nm, and the IR wavelength may be between about 800 nm and about 1000 nm. It will be understood that light source 130 may include any number of light sources with any suitable characteristics. In embodiments where an array of sensors is used in place of single sensor 102, each sensor may be configured to emit a single wavelength. For example, a first sensor may emit only a red light while a second may emit only an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 140 may be chosen to be specifically sensitive to the chosen targeted energy spectrum of light source 130.

In some embodiments, detector 140 may be configured to detect the intensity of light at the red and IR wavelengths. In some embodiments, an array of sensors may be used and each sensor in the array may be configured to detect an intensity of a single wavelength. In operation, light may enter detector 140 after passing through the subject's tissue. Detector 140 may convert the intensity of the received light into an electrical signal. The light intensity may be directly related to the absorbance and/or reflectance of light in the tissue. That is, when more light at a certain wavelength is absorbed or reflected, less light of that wavelength is received from the tissue by detector 140. After converting the received light to an electrical signal, detector 140 may send the detection signal to monitor 104, where the detection signal may be processed and physiological parameters may be determined (e.g., based on the absorption of the red and IR wavelengths in the subject's tissue). In some embodiments, the detection signal may be preprocessed by sensor 102 before being transmitted to monitor 104.

In the embodiment shown, monitor 104 includes control circuit 110, light drive circuit 120, front end processing circuit 150, back end processing circuit 170, user interface 180, and communication interface 190. Monitor 104 may be communicatively coupled to sensor 102.

Control circuit 110 may be coupled to light drive circuit 120, front end processing circuit 150, and back end processing circuit 170, and may be configured to control the operation of these components. In some embodiments, control circuit 110 may be configured to provide timing control signals to coordinate their operation. For example, light drive circuit 120 may generate a light drive signal, which may be used to turn on and off the light source 130, based on the timing control signals. The front end processing circuit 150 may use the timing control signals to operate synchronously with light drive circuit 120. For example, front end processing circuit 150 may synchronize the operation of an analog-to-digital converter and a demultiplexer with the light drive signal based on the timing control signals. In addition, the back end processing circuit 170 may use the timing control signals to coordinate its operation with front end processing circuit 150.

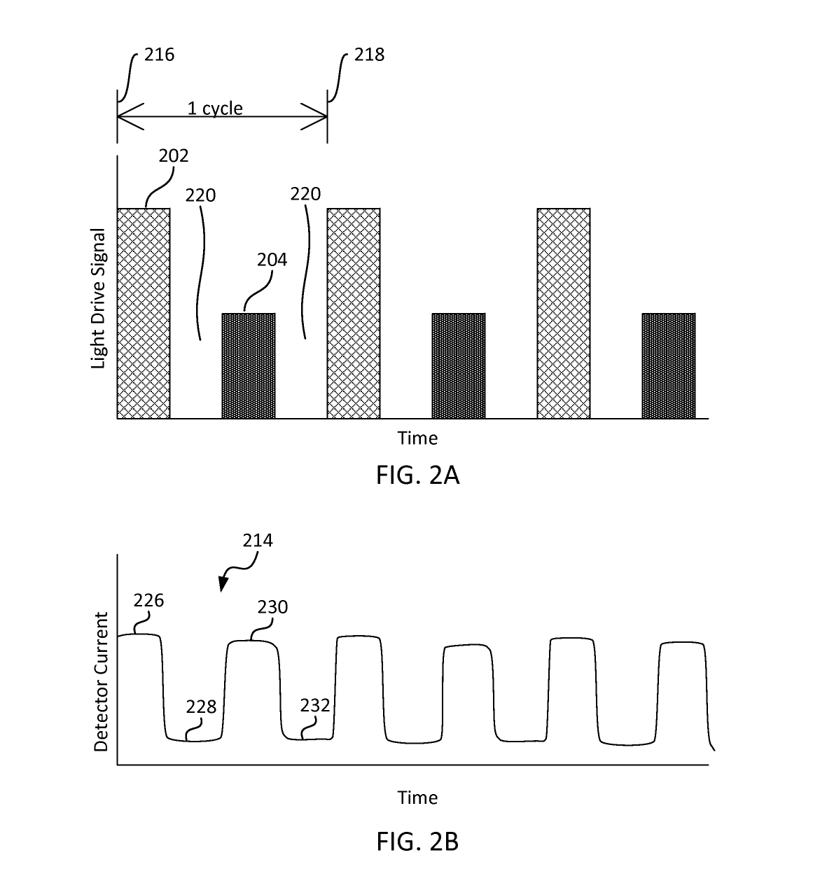

Light drive circuit 120, as discussed above, may be configured to generate a light drive signal that is provided to light source 130 of sensor 102. Light drive circuit 120 may comprise an amplifier, a voltage sources, a current source, any other suitable power supply, or any suitable combination thereof. The light drive signal may, for example, control the intensity of light source 130 and the timing of when light source 130 is turned on and off. When light source 130 is configured to emit two or more wavelengths of light, the light drive signal may be configured to control the operation of each wavelength of light. The light drive signal may comprise a single signal or may comprise multiple signals (e.g., one signal for each wavelength of light). In some embodiments, the light drive signal may provide different power for each wavelength of light such that the detected signal amplitudes are similar for each wavelength of light. An illustrative light drive signal is shown in FIG. 2A.

In some embodiments, control circuit 110 and light drive circuit 120 may generate light drive parameters based on a metric. For example, back end processing 170 may receive information about received light signals, determine light drive parameters based on that information, and send corresponding information to control circuit 110.

One or more components of system 100 may be referred to as front end channel circuit 151. In some embodiments, the front end channel circuit 151 may comprise detector 140 and front-end processing circuit 150 as well as any electronic and other types of connections between detector 140 and front-end processing circuit 150. In some embodiments, front end channel circuit 151 may comprise any other combination of components of system 100. The front end channel circuit 151 may be configured to receive light generated by the light source 130, and generate an at least one signal responsive to receiving the light. The front end channel circuit 151 may comprise a response time corresponding to its components. For example, the response time of the front end channel circuit 151 may comprise the response time of the combination of detector 140, front-end processing circuit 150 and the connection between detector 140 and front-end processing circuit 150. The response time of front-end processing circuit 150, and thus front end channel circuit 151, may include the response time of any part of the front-end processing circuit 150, such as the analog conditioning 152 and digital conditioning 158. Stated another way, the response time of the front end channel circuit 151 may comprise the settling time of the electronic circuits of detector 140 and front end channel circuit 151. In some embodiments, front end circuit 151 may not include detector 140 and the connection between detector 140 and front-end processing circuit 150. The response time may be advantageously used in order to generate a desired analog signal using techniques describes above and below.

FIG. 2A shows an illustrative plot of a light drive signal including red light drive pulse 202 and IR light drive pulse 204 in accordance with some embodiments of the present disclosure. Light drive pulses 202 and 204 are illustrated as square waves. These pulses may include shaped waveforms rather than a square wave. The shape of the pulses may be generated by a digital signal generator, digital filters, analog filters, any other suitable equipment, or any combination thereof. For example, light drive pulses 202 and 204 may be generated by light drive circuit 120 under the control of control circuit 110. As used herein, drive pulses may refer to the high and low states of a shaped pulse, switching power or other components on and off, high and low output states, high and low values within a continuous modulation, other suitable relatively distinct states, or any combination thereof. The light drive signal may be provided to light source 130, including red light drive pulse 202 and IR light drive pulse 204 to drive red and IR light emitters, respectively, within light source 130. Red light drive pulse 202 may have a higher amplitude than IR light drive pulse 204 since red LEDs may be less efficient than IR LEDs at converting electrical energy into light energy. In some embodiments, the output levels may be equal, may be adjusted for nonlinearity of emitters, may be modulated in any other suitable technique, or any combination thereof. Additionally, red light may be absorbed and scattered more than IR light when passing through perfused tissue.

When the red and IR light sources are driven in this manner they emit pulses of light at their respective wavelengths into the tissue of a subject in order generate sensor signals that include physiological information that physiological monitoring system 100 may process to calculate physiological parameters. It will be understood that the light drive amplitudes of FIG. 2A are merely exemplary and that any suitable amplitudes or combination of amplitudes may be used, and may be based on the light sources, the subject tissue, the determined physiological parameter, modulation techniques, power sources, any other suitable criteria, or any combination thereof.

The light drive signal of FIG. 2A may also include “off” periods 220 between the red and IR light drive pulse. “Off” periods 220 are periods during which no drive current may be applied to light source 130. “Off” periods 220 may be provided, for example, to prevent overlap of the emitted light, since light source 130 may require time to turn completely on and completely off. The period from time 216 to time 218 may be referred to as a drive cycle, which includes four segments: a red light drive pulse 202, followed by an “off” period 220, followed by an IR light drive pulse 204, and followed by an “off” period 220. After time 218, the drive cycle may be repeated (e.g., as long as a light drive signal is provided to light source 130). It will be understood that the starting point of the drive cycle is merely illustrative and that the drive cycle can start at any location within FIG. 2A, provided the cycle spans two drive pulses and two “off” periods. Thus, each red light drive pulse 202 and each IR light drive pulse 204 may be understood to be surrounded by two “off” periods 220. “Off” periods may also be referred to as dark periods, in that the emitters are dark or returning to dark during that period. It will be understood that the particular square pulses illustrated in FIG. 2A are merely exemplary and that any suitable light drive scheme is possible. For example, light drive schemes may include shaped pulses, sinusoidal modulations, time division multiplexing other than as shown, frequency division multiplexing, phase division multiplexing, any other suitable light drive scheme, or any combination thereof.

Referring back to FIG. 1, front end processing circuit 150 may receive a detection signal from detector 140 and provide one or more processed signals to back end processing circuit 170. The term “detection signal,” as used herein, may refer to any of the signals generated within front end processing circuit 150 as it processes the output signal of detector 140. Front end processing circuit 150 may perform various analog and digital processing of the detector signal. One suitable detector signal that may be received by front end processing circuit 150 is shown in FIG. 2B.

FIG. 2B shows an illustrative plot of detector current waveform 214 that may be generated by a sensor in accordance with some embodiments of the present disclosure. The peaks of detector current waveform 214 may represent current signals provided by a detector, such as detector 140 of FIG. 1, when light is being emitted from a light source. The amplitude of detector current waveform 214 may be proportional to the light incident upon the detector. The peaks of detector current waveform 214 may be synchronous with drive pulses driving one or more emitters of a light source, such as light source 130 of FIG. 1. For example, detector current peak 226 may be generated in response to a light source being driven by red light drive pulse 202 of FIG. 2A, and peak 230 may be generated in response to a light source being driven by IR light drive pulse 204. Valley 228 of detector current waveform 214 may be synchronous with periods of time during which no light is being emitted by the light source, or the light source is returning to dark, such as “off” period 220. While no light is being emitted by a light source during the valleys, detector current waveform 214 may not fall all of the way to zero.

It will be understood that detector current waveform 214 may be an at least partially idealized representation of a detector signal, assuming perfect light signal generation, transmission, and detection. It will be understood that an actual detector current will include amplitude fluctuations, frequency deviations, droop, overshoot, undershoot, rise time deviations, fall time deviations, other deviations from the ideal, or any combination thereof. It will be understood that the system may shape the drive pulses shown in FIG. 2A in order to make the detector current as similar as possible to idealized detector current waveform 214.

Referring back to FIG. 1, front end processing circuit 150, which may receive a detection signal, such as detector current waveform 214, may include analog conditioning 152, analog-to-digital converter (ADC) 154, demultiplexer 156, digital conditioning 158, decimator/interpolator 160, ambient subtractor 162, and a down-converter (not shown).

Analog conditioning 152 may perform any suitable analog conditioning of the detector signal. The conditioning performed may include any type of filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. Analog conditioning 152 may increase the response time of front end channel circuit 151.

The conditioned analog signal may be processed by analog-to-digital converter 154, which may convert the conditioned analog signal into a digital signal. Analog-to-digital converter 154 may operate under the control of control circuit 110. Analog-to-digital converter 154 may use timing control signals from control circuit 110 to determine when to sample the analog signal. Analog-to-digital converter 154 may be any suitable type of analog-to-digital converter of sufficient resolution to enable a physiological monitor to accurately determine physiological parameters. In some embodiments, the front end processing circuit 150 may be configured to take advantage of the full dynamic range of analog-to-digital converter 154. This may be achieved by applying gain to the analog signal, by analog conditioning 152, to map the expected range of the signal to the full or close to full output range of analog-to-digital converter 154.

Demultiplexer 156 may operate on the analog or digital form of the detector signal to separate out different components of the signal. For example, detector current waveform 214 of FIG. 2B includes a red component corresponding to peak 226, an IR component corresponding to peak 230, and at least one ambient component corresponding to valley 230. Demultiplexer 156 may operate on detector current waveform 214 of FIG. 2B to generate a red signal, an IR signal, a first ambient signal (e.g., corresponding to the ambient component corresponding to valley 228 that occurs immediately after the peak 226), and a second ambient signal (e.g., corresponding to the ambient component corresponding to valley 232 that occurs immediately after the peak 230). Demultiplexer 156 may operate under the control of control circuit 110. For example, demultiplexer 156 may use timing control signals from control circuit 110 to identify and separate out the different components of the detector signal.

Digital conditioning 158 may perform any suitable digital conditioning of the detector signal. Digital conditioning 158 may include any type of digital filtering of the signal (e.g., low pass, high pass, band pass, notch, or any other suitable filtering), amplifying, performing an operation on the signal, performing any other suitable digital conditioning, or any combination thereof. Digital conditioning 158 may increase the response time of front end channel circuit 151.

Decimator/interpolator 160 may decrease the number of samples in the digital detector signal. For example, decimator/interpolator 160 may decrease the number of samples by removing samples from the detector signal or replacing samples with a smaller number of samples. The decimation or interpolation operation may include or be followed by filtering to smooth the output signal.

Ambient subtractor 162 may operate on the digital signal. In some embodiments, ambient subtractor 162 may remove dark or ambient contributions to the received signal.

The down-converter may operate on the conditioned analog signal. In some embodiments, the down-converter may mix or down convert the conditioned analog signal to reduce modulated frequency of the conditioned analog signal to intermediate frequency (IF) or baseband frequency before analog-to-digital conversion.

The components of front end processing circuit 150 are merely illustrative and any suitable components and combinations of components may be used to perform the front end processing operations.

Back end processing circuit 170 may include processor 172 and memory 174. Processor 172 may be adapted to execute software, which may include an operating system and one or more applications, as part of performing the functions described herein. Processor 172 may receive and further process sensor signals received from front end processing circuit 150. For example, processor 172 may determine one or more physiological parameters based on the received physiological signals. Processor 172 may include an assembly of analog or digital electronic components. Processor 172 may calculate physiological information. For example, processor 172 may compute one or more of blood oxygen saturation (e.g., arterial, venous, or both), pulse rate, respiration rate, respiration effort, blood pressure, hemoglobin concentration (e.g., oxygenated, deoxygenated, and/or total), any other suitable physiological parameters, or any combination thereof. Processor 172 may perform any suitable signal processing of a signal, such as any suitable band-pass filtering, adaptive filtering, closed-loop filtering, any other suitable filtering, and/or any combination thereof. Processor 172 may also receive input signals from additional sources not shown. For example, processor 172 may receive an input signal containing information about treatments provided to the subject from user interface 180. Additional input signals may be used by processor 172 in any of the calculations or operations it performs in accordance with back end processing circuit 170 or monitor 104.

Memory 174 may include any suitable computer-readable media capable of storing information that can be interpreted by processor 172. In some embodiments, memory 174 may store calculated values such as light drive parameter limits. Calculated values may be stored in a memory device for later retrieval. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other non-transitory medium which can be used to store the desired information and which can be accessed by components of the system. Back end processing circuit 170 may be communicatively coupled with user interface 180 and communication interface 190.

User interface 180 may include user input 182, display 184, and speaker 186. User interface 180 may include, for example, any suitable device such as one or more medical devices (e.g., a medical monitor that displays various physiological parameters, a medical alarm, or any other suitable medical device that either displays physiological parameters or uses the output of back end processing 170 as an input), one or more display devices (e.g., monitor, personal digital assistant (PDA), mobile phone, tablet computer, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

User input 182 may include any type of user input device such as a keyboard, a mouse, a touch screen, buttons, switches, a microphone, a joy stick, a touch pad, or any other suitable input device. The inputs received by user input 182 can include information about the subject, such as age, weight, height, diagnosis, medications, treatments, and so forth.

In some embodiments, the subject may be a medical patient and display 184 may exhibit a list of values which may generally apply to the patient, such as, for example, age ranges or medication families, which the user may select using user input 182. Additionally, display 184 may display, for example, an estimate of a subject's blood oxygen saturation generated by monitor 104 (e.g., an “SpO₂” measurement), pulse rate information, respiration rate and/or effort information, blood pressure information, hemoglobin concentration information, any other parameters, and any combination thereof. Display 184 may include any type of display such as a cathode ray tube display, a flat panel display such as a liquid crystal display or plasma display, or any other suitable display device. Speaker 186 within user interface 180 may provide an audible sound that may be used in various embodiments, such as for example, sounding an audible alarm in the event that a patient's physiological parameters are not within a predefined normal range.

Communication interface 190 may enable monitor 104 to exchange information with external devices. Communications interface 190 may include any suitable hardware, software, or both, which may allow monitor 104 to communicate with electronic circuitry, a device, a network, a server or other workstations, a display, or any combination thereof. Communications interface 190 may include one or more receivers, transmitters, transceivers, antennas, plug-in connectors, ports, communications buses, communications protocols, device identification protocols, any other suitable hardware or software, or any combination thereof. Communications interface 190 may be configured to allow wired communication (e.g., using USB, RS-232, Ethernet, or other standards), wireless communication (e.g., using WiFi, IR, WiMax, BLUETOOTH, USB, or other standards), or both. For example, communications interface 190 may be configured using a universal serial bus (USB) protocol (e.g., USB 2.0, USB 3.0), and may be configured to couple to other devices (e.g., remote memory devices storing templates) using a four-pin USB standard Type-A connector (e.g., plug and/or socket) and cable. In some embodiments, communications interface 190 may include an internal bus such as, for example, one or more slots for insertion of expansion cards.

It will be understood that the components of physiological monitoring system 100 that are shown and described as separate components are shown and described as such for illustrative purposes only. In some embodiments the functionality of some of the components may be combined in a single component. For example, the functionality of front end processing circuit 150 and back end processing circuit 170 may be combined in a single processor system. Additionally, in some embodiments the functionality of some of the components of monitor 104 shown and described herein may be divided over multiple components. For example, some or all of the functionality of control circuit 110 may be performed in front end processing circuit 150, in back end processing circuit 170, or both. In other embodiments, the functionality of one or more of the components may be performed in a different order or may not be required. In some embodiments, all of the components of physiological monitoring system 100 can be realized in processor circuit.

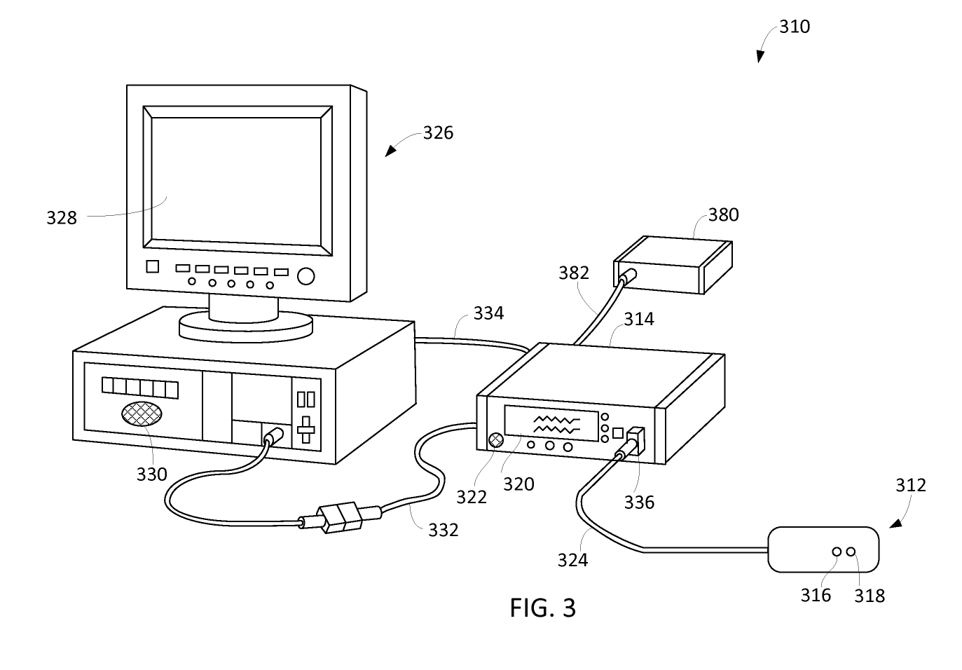

FIG. 3 is a perspective view of an illustrative physiological monitoring system 310 in accordance with some embodiments of the present disclosure. In some embodiments, one or more components of physiological monitoring system 310 may include one or more components of physiological monitoring system 100 of FIG. 1. Physiological monitoring system 310 may include sensor unit 312 and monitor 314. In some embodiments, sensor unit 312 may be part of an oximeter. Sensor unit 312 may include one or more light source 316 for emitting light at one or more wavelengths into a subject's tissue. One or more detector 318 may also be provided in sensor unit 312 for detecting the light that is reflected by or has traveled through the subject's tissue. Any suitable configuration of light source 316 and detector 318 may be used. In some embodiments, sensor unit 312 may include multiple light sources and detectors, which may be spaced apart. Physiological monitoring system 310 may also include one or more additional sensor units (not shown) that may, for example, take the form of any of the embodiments described herein with reference to sensor unit 312. An additional sensor unit may be the same type of sensor unit as sensor unit 312, or a different sensor unit type than sensor unit 312 (e.g., a photoacoustic sensor). Multiple sensor units may be capable of being positioned at two different locations on a subject's body.

In some embodiments, sensor unit 312 may be connected to monitor 314 as shown. Sensor unit 312 may be powered by an internal power source, e.g., a battery (not shown). Sensor unit 312 may draw power from monitor 314. In some embodiments, the sensor may be wirelessly connected (not shown) to monitor 314. Monitor 314 may be configured to calculate physiological parameters based at least in part on data relating to light emission and light detection received from one or more sensor units such as sensor unit 312. For example, monitor 314 may be configured to determine pulse rate, respiration rate, respiration effort, blood pressure, blood oxygen saturation (e.g., arterial, venous, or both), hemoglobin concentration (e.g., oxygenated, deoxygenated, and/or total), any other suitable physiological parameters, or any combination thereof. In some embodiments, calculations may be performed on the sensor units or an intermediate device and the result of the calculations may be passed to monitor 314. Further, monitor 314 may include display 320 configured to display the physiological parameters or other information about the system. In the embodiment shown, monitor 314 may also include a speaker 322 to provide an audible sound that may be used in various other embodiments, such as for example, sounding an audible alarm in the event that a subject's physiological parameters are not within a predefined normal range. In some embodiments, physiological monitoring system 310 may include a stand-alone monitor in communication with the monitor 314 via a cable or a wireless network link. In some embodiments, monitor 314 may be implemented as display 184 of FIG. 1.

In some embodiments, sensor unit 312 may be communicatively coupled to monitor 314 via a cable 324 at port 336. Cable 324 may include electronic conductors (e.g., wires for transmitting electronic signals from detector 318), optical fibers (e.g., multi-mode or single-mode fibers for transmitting emitted light from light source 316), any other suitable components, any suitable insulation or sheathing, or any combination thereof. In some embodiments, a wireless transmission device (not shown) or the like may be used instead of or in addition to cable 324. Monitor 314 may include a sensor interface configured to receive physiological signals from sensor unit 312, provide signals and power to sensor unit 312, or otherwise communicate with sensor unit 312. The sensor interface may include any suitable hardware, software, or both, which may be allow communication between monitor 314 and sensor unit 312.

In some embodiments, physiological monitoring system 310 may include calibration device 380. Calibration device 380, which may be powered by monitor 314, a battery, or by a conventional power source such as a wall outlet, may include any suitable calibration device. Calibration device 380 may be communicatively coupled to monitor 314 via communicative coupling 382, and/or may communicate wirelessly (not shown). In some embodiments, calibration device 380 is completely integrated within monitor 314. In some embodiments, calibration device 380 may include a manual input device (not shown) used by an operator to manually input reference signal measurements obtained from some other source (e.g., an external invasive or non-invasive physiological measurement system).

In the illustrated embodiment, physiological monitoring system 310 includes a multi-parameter physiological monitor 326. The monitor 326 may include a cathode ray tube display, a flat panel display (as shown) such as a liquid crystal display (LCD) or a plasma display, or may include any other type of monitor now known or later developed. Multi-parameter physiological monitor 326 may be configured to calculate physiological parameters and to provide a display 328 for information from monitor 314 and from other medical monitoring devices or systems (not shown). For example, multi-parameter physiological monitor 326 may be configured to display an estimate of a subject's blood oxygen saturation and hemoglobin concentration generated by monitor 314. Multi-parameter physiological monitor 326 may include a speaker 330.

Monitor 314 may be communicatively coupled to multi-parameter physiological monitor 326 via a cable 332 or 334 that is coupled to a sensor input port or a digital communications port, respectively and/or may communicate wirelessly (not shown). In addition, monitor 314 and/or multi-parameter physiological monitor 326 may be coupled to a network to enable the sharing of information with servers or other workstations (not shown). Monitor 314 may be powered by a battery (not shown) or by a conventional power source such as a wall outlet.

In some embodiments, any of the processing components and/or circuits, or portions thereof, of FIGS. 1 and 3, including sensors 102 and 312 and monitors 104, 314, and 326, may be referred to collectively as processing equipment. In some embodiments, all or some of the components of the processing equipment may be referred to as a processing module.

In some embodiments, the present disclosure may be embodied by a physiological monitoring system. In some embodiments, the physiological monitoring system (such as system 100 of FIG. 1) is a PPG system comprising a blood oximeter that is configured to measure, for example, oxygen saturation, pulse, and/or blood pleasure of a subject. In some embodiments, the PPG system may comprise a light drive circuit (e.g., light drive circuit 120 of FIG. 1), a light source (e.g., light source 130 of FIG. 1) that may be controlled by a light drive circuit (e.g., light drive circuit of 120 of FIG. 1), and a front end channel (e.g., front end channel circuit 151 of FIG. 1) configured to receive light generated by the light source. The light source may comprise one or more light sources, such as multiple LEDs configured, e.g., as described with respect to light source 130 of FIG. 1 above.

In some embodiments, the light drive circuit may be configured to provide a digital signal to the light source. The light source may comprise red and IR light emitting diodes (LEDs). Alternatively, light source may comprise any number of LEDs and/or any number of other light producing elements, devices or systems. The digital signal may be stored by the physiological monitoring system. For example, the digital signal may be stored by the control circuit 110 of FIG. 1 on a non-transitory computer-readable medium. Alternatively, the digital signal may be dynamically generated by the physiological monitoring system. For example, the digital signal may be generated by a back end processing circuit (e.g., back end processing circuit 170 of FIG. 1) that comprises a processor (e.g., processor 172 of FIG. 1) and non-transitory computer readable memory (e.g., memory 174 of FIG. 1) that may comprise computer readable instructions that are executable by the processor. Alternatively, the digital signal may be generated by a front end processing circuit that comprises a second processor and second non-transitory computer readable memory that may comprise computer readable instructions that are executable by the second processor. In some embodiments, the digital signal may be based on a desired analog signal. For example, the digital signal may be a signal that is a representation, an approximation, or an encoding of the desired analog signal. The digital signal may represent, approximate, or encode the desired analog signal using a series of two or more discrete states connected by rising and falling edges. In some embodiments, the digital signal may comprise pulses. For example, the digital signal may comprise square wave pulses that define high states and low states distributed across the digital signal in a predetermined manner. For example, the digital signal may be a PWM version of a desired analog signal.

The light drive circuit may be configured to provide power to the light source during each pulse of the digital signal. The light drive circuit may also be configured to provide no power (or reduced power) to the light source during the intervals between pulses. For example, if the digital signal comprises square waves, the light drive circuit may provide power to the light source during the high states of the digital signal and provide no power (or reduced power) during the low states of the digital signal. In some embodiments, the light drive circuit may provide fixed voltage or fixed current power during the high states of the digital signal. In some embodiments, the light drive circuit may provide two or more discrete levels of voltage based on an at least one of the adjustable features of the pulses (e.g., pulse amplitude), or based on any other factor.

In some embodiments, the pulses may comprise at least one adjustable feature. The power (e.g., average power) provided to the light source may be varied by the light drive circuit based on the adjustable feature of the digital signal. In some embodiments, the power may be varied in accordance with changes in the adjustable feature of the pulses. The adjustable feature of the pulses the may comprise the width of the pulses when the digital signal is a PWM signal. Accordingly, in some embodiments, the power provided to the light source by the light drive circuit may vary in accordance with the variation in the width of the pulses of the digital signal. For example, the digital signal may comprise square wave pulses, where the high states of the square wave pulses have varying widths. In some embodiments, the light drive circuit may provide power to the light source during the plurality of high states of the digital signal, such that the time period during which the power is provided to the light source corresponds to the widths of the high states of the digital signal.

In some embodiments, the adjustable feature of the digital signal may be one or more of the frequency of the plurality of pulses, density of the plurality of pulses, amplitude of the plurality of pulses, positions of the pulses or pulse code of the pulses. In each of these embodiments, the light drive circuit may vary the power provided to the light sources in accordance with the adjustable feature of the plurality of pulses.

The front end channel of an exemplary system may be configured to receive light generated by the light source. In some embodiments, the light received by the front end channel may be attenuated by body tissue of a subject. The front end channel may comprise a processing circuit, for example, a printed circuit board corresponding to front end processing circuit 150 of FIG. 1. The front end channel may also comprise a light detector connected to the processing circuit. The light detector may be configured to detect the presence of light and generate an electric signal that is transmitted to the front end processing circuit in response to detecting light. The processing circuit may be configured to process the electric signals received from the detector. In some embodiments, the front end channel may comprise a response time. In some embodiments the response time of the front end channel may comprise the response time of the processing circuit with respect to the electric signal received from the detector. In some embodiments, the response time of the front end channel may additionally comprise the response time of the detector, and/or the response time of the connection between the detector and the processing circuit.

In some embodiments, the front end channel may receive pulses generated by the light source. These pulses may be generated by the light source as a result of the light drive circuit providing power to the light source according to the adjustable digital signal. In some embodiments, the digital signal may comprise pulses generated at one or more frequencies. The one or more frequencies may be considered to have one or more corresponding periods. In some embodiments, the periods of the digital signal may be substantially shorter than the response time of the front end channel. For example, the pulse frequency may be 16 Mhz and the corresponding period may be 0.0000000625 seconds, while the response time of the front end channel may comprise a time period that is at least a ten times longer than the corresponding period. In some embodiments, the response time may be longer than the pulse period by any multiple and/or factor. In some embodiments, other pulse frequencies and other response times may also be used. In particular, any pulse frequency with an associated period that is substantially shorter than the response time of the front end channel may be used.

In some embodiments, the widths of the digital pulses received by the front end channel may be substantially shorter than the response time of the front end channel. In some embodiments, the front end channel may effectively integrate multiple digital pulses in order create an analog pulse. In some embodiments, the digital signal may be constructed in such a fashion that digital pulses, when integrated by the front end channel, will yield an approximated desired analog pulse (e.g., a sinusoid wave or a shaped square wave). In some embodiments, the front end channel may effectively integrate the digital pulses due to the front end channel having a longer response time than the width of the digital pulses. In some embodiments, the front end channel may not have enough time to settle after responding to a first pulse of the digital pulses before the second pulse of the digital pulses received. Consequently, the signal generated by the front end channel may increase during the pulses of the digital signal and begin to attenuate (but not completely attenuate) between the pulses of the digital signal, thus producing an integrated analog signal.

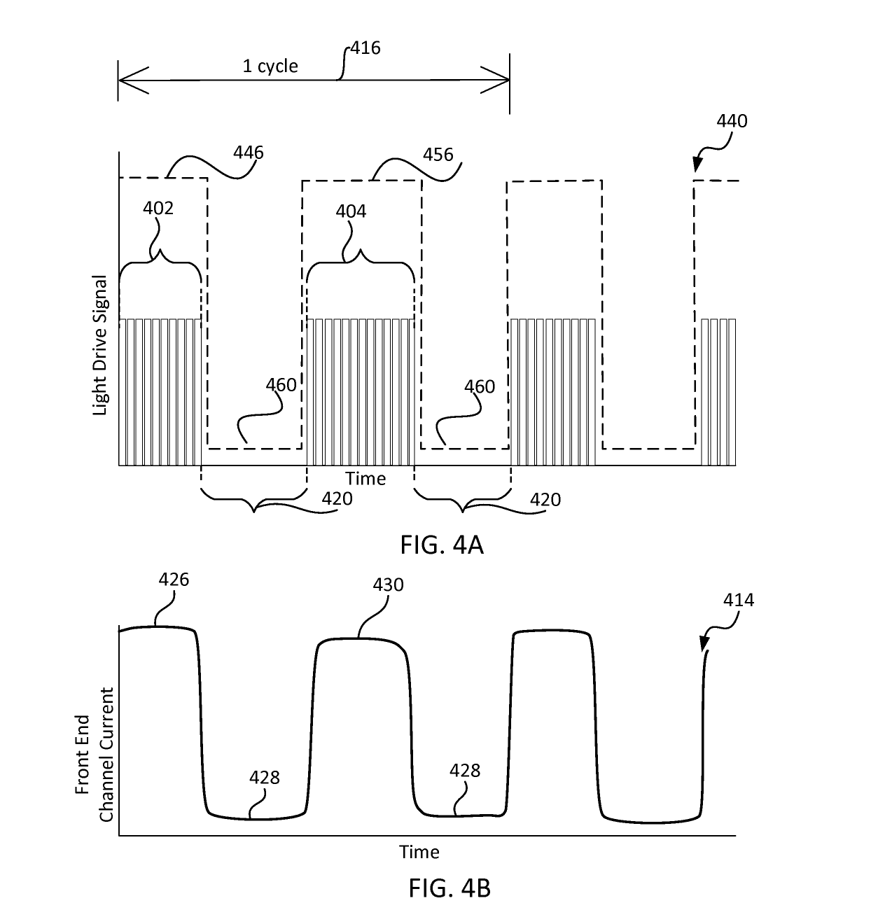

FIG. 4A shows an illustrative plot of a desired signal and of a light drive signal generated based on the desired signal in accordance with some embodiments of the present disclosure. The light drive signal illustrated by FIG. 4A may be provided by a light drive circuit (e.g., light drive circuit 120 of FIG. 1) to a light source (e.g., light source 130 of FIG. 1) in accordance with some exemplary embodiments of a PPG system (e.g., system 100 of FIG. 1). FIG. 4A shows a desired signal 440 (in a dashed line) that may comprise a repeating series of idealized square waves 446 and 456. The desired signal 440 may also include dark periods 460. In some embodiments, the light drive signal may comprise light drive pulses 402, light drive pulses 404, and dark periods 420 that may comprise a digital representation or encoding of the desired signal 440.

In some embodiments, the light drive signal may comprise light drive pulses 402 and light drive pulses 404 that are a plurality of square waves comprising high states of the light drive signal. The portions of the light drive signal between light drive pulses 402 and light drive pulses 404 comprise low states of the light drive signal. In some embodiments, light drive pulses 402 may regulate power provided to a red LED, while light drive pulses 404 may regulate power provided to an IR LED. A full signal cycle 416 may comprise light drive pulses 402 followed by dark period 420, followed by light drive pulses 404, followed by another dark period 420. The full signal cycle 416 may be repeated for any time period, or until the light drive is turned off or unpowered. Light drive pulses 402 may represent a digitally modulated version (e.g., a PWM version) of idealized square wave 446. Light drive pulses 404 may represent a digitally modulated version (e.g., a PWM version) of idealized square wave 456.

In the embodiment illustrated by FIG. 4A, light drive pulses 402 and light drive pulses 404 may comprise a Pulse Width Modulated (PWM) version of desired signal 440. PWM modulated light drive pulses 402 and light drive pulses 404 may comprise pulses that have fixed pulse frequency and amplitude. The light drive pulses 402 and light drive pulses 404 may comprise a variable pulse width (i.e., duration). In some embodiments, the light drive pulses 402 and light drive pulses 404 may comprise a different variable feature (e.g., pulse amplitude, pulse density) or any combination of variable features. In some embodiments, the light drive pulses 402 and 404 may be generated by a digital signal generator. For example, light drive pulses 402 and light drive pulses 404 may be generated by light drive circuit 120 under the control of control circuit 110 of FIG. 1.

In some embodiments, PWM modulated light drive pulses 402 and light drive pulses 404 may comprise right-aligned, left-aligned or center-aligned pulses. Left-aligned pulses may comprise left edges that are aligned in time. Right-aligned pulses may comprise right edges that are aligned in time. Center-aligned pulses may comprise pulse centers that are aligned in time. In some embodiments, the pulses may be aligned using a counter that is incremented based on a clock of a processor. In some embodiments (e.g., when desired signal 440 comprises shaped pulses), center-aligned pulses may reduce noise in the signal generated by the front end channel circuit, by providing non-uniformly distributed right edges and left edges. In some embodiments, the use of right-aligned or left-aligned PWM pulses may change the shape or position of the desired waveform, which can cause a phase change in the signal produced by the front-end channel. This phase change may affect the separation and processing of the red LED and IRLED light signals. However, the use of a center aligned PWM pulses, where the centers of the pulses are aligned and the edges of the pulses move, may reduce or eliminate phase changes in the desired signal.

In some embodiments, a light drive circuit (e.g., light drive circuit 120 of FIG. 1) may provide power to the light sources (e.g., light source 130) according to the width of light drive pulses 402 and light drive pulses 404. For example, the light drive circuit may apply a fixed voltage or fixed current to the red LED during light drive pulses 402. The light drive circuit may also apply a fixed voltage or fixed current to the IR LED during light drive pulses 404. The light drive circuit may apply no voltage (or very low voltage) to any of the light sources during the portions of the light drive signal between light drive pulses 402 and light drive pulses 404 and during the dark periods 420. In some embodiments, the light drive circuit may provide a fixed voltage (e.g., 1.8V) or fixed current to a light source during light drive pulses 402 and light drive pulses 404. In other embodiments, the light drive circuit may provide power to the light sources according to a different variable feature of drive pulses 402 and light drive pulses 404, or according to any combination of features. For example, the light drive circuit may provide a variable voltage at two or more discrete levels in accordance with varying amplitude of the light drive pulses 402 and light drive pulses 404.

The use of a light drive signal that comprises digitally modulated (e.g., PWM) light drive pulses allows the times during which a light source is turned on to vary, which, in turn, varies the power consumed by the light source. Operating a light source, such as an LED, at a fixed voltage (or fixed current) may be advantageous because the light source may be more power efficient when not subject to resistive losses required to adjust the voltage. In additions, when an LED is driven over a large linear dynamic range by variable voltage, shifts in spectral content may occur which may be undesirable. These nonlinear effects may be disadvantageous to PPG systems, for example, pulse oximeters, that determine oxygen saturation based on calibration coefficients that correspond to the wavelengths of light used. Accordingly, in some embodiments, a PWM technique using fixed voltage (or fixed current) may be advantageous when compared to providing power to a light source using variable voltage.

Light sources (e.g., light source 130) may have a fast response time. In particular, light sources that comprise one or more LEDs may respond very quickly to provided power. Accordingly, in some embodiments, power may be provided to a light source according to a light drive signal that comprises high frequency square waves. The light source may then generate light corresponding to the provided square waves. Unlike the light source, an exemplary front end channel circuit of the PPG system may be configured to have a relatively slow response time, such that square waves of the light waveform received at the front end channel circuit (e.g., front end processing circuit 151) may be effectively integrated by the front end channel circuit.

FIG. 4B shows an illustrative plot of a signal generated by a front end channel circuit (e.g., front end channel circuit 151) in accordance with some embodiments of the present disclosure. In the shown embodiment, FIG. 4B illustrates one exemplary front end channel signal 414 (e.g., output current of front end channel circuit 151) that may be generated by the front end channel circuit in response to receiving light from a light source that received power according to light drive pulses 402 and light drive pulses 404. As explained above, light drive pulses 402 and light drive pulses 404 may represent a digitally modulated version of desired signal 440. The light generated by the light source in response to receiving light drive pulses 402 and light drive pulses 404 may be received by a detector and processed by the front end channel circuit. In a way, the combination of the light drive circuit, the light source and the front end channel circuit act like digital to analog converter. The light drive pulses 402 and light drive pulses 404 may be considered to comprise a digital signal, while the resulting front end channel signal 414 created by the front end channel circuit may be considered to comprise an analog signal.

The resulting front end channel signal 414 may be approximately the same as desired signal 440. In some embodiments, due to signal integration performed by the front end channel circuit, the front end channel signal 414 may increase during each of light drive pulses 402 and light drive pulses 404, and be attenuated somewhat (but not completely) during portions of the light drive signal between light drive pulses 402 and light drive pulses 404. In some embodiments, the resulting front end channel signal 414 may be a saw-tooth shaped approximation of desired signal 440 due to integration by the front end channel circuit of the received light. In some embodiments, light drive pulses 402 and light drive pulses 404 may have a very high pulse frequency improving the quality of the approximation of the resulting front end channel signal 414. In some embodiments, the resulting front end channel signal 414 may comprise square waves (e.g., square waves 426 and 430) that approximate the idealized square waves of desired signal 440, but contain imperfections due to amplitude fluctuations, frequency deviations, droop, overshoot, undershoot, rise time deviations, fall time deviations, other deviations from the ideal, or any combination thereof.

It should be noted that the light drive pulses shown by FIG. 4A are given as an example only, and that in some embodiments the light drive pulses 402 and light drive pulses 404 may represented a digital signal modulated at a much higher frequency (e.g., 16 Mhz). In such embodiments, each of the light drive signals 402 or light drive pulses 404 may comprise a relatively large number of pulses (e.g., 16,000 pulses) rather than the smaller number of pulses that are shown by FIG. 4A. In some embodiments, light drive pulses 402 and light drive pulses 404 may comprise any other number of constant or variable frequencies and/or periods.

In the embodiment demonstrated by FIG. 4B, the resulting integrated front end channel signal 414 may comprise square wave 426 representative of the light generated by a red LED of the light source and square wave 430 representative of the light generated by a IR LED of the light source. The square waves 426 and 430 may have a period that is substantially longer than the period of the light drive pulses 402 and light drive pulses 404. The integrated front end channel signal 414 may also comprise dark periods 428 during which no power (or reduced power) was provided to the light source.

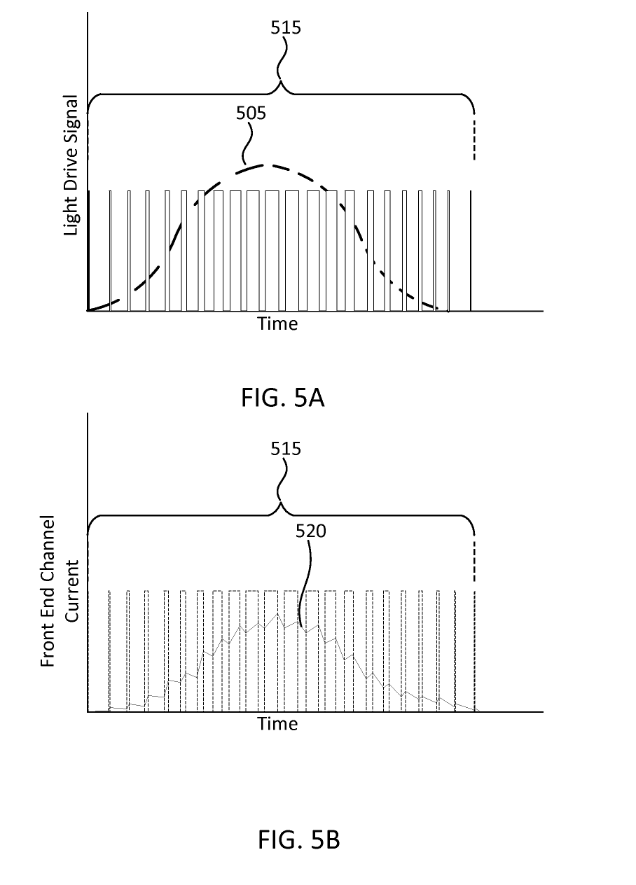

FIG. 5A shows an illustrative plot of an exemplary light drive signal in accordance with some embodiments of the present disclosure. For example, FIG. 5A illustrates a light drive signal comprising square wave light drive pulses 515 that may be used to provide power to one or more light sources (e.g., light source 130) of a physiological monitoring system (e.g., system 100) in accordance with some embodiments described above. In some embodiments, light drive pulses 515 may comprise a digital representation (e.g., Pulse Width Modulated digital representation) of a desired analog signal 505. It should be noted that figure FIG. 5A labels square wave light drive pulses 515 collectively with a curly bracket. The curly bracket is not intended to refer to desired analog signal 505. In the shown embodiment, a physiological monitoring system may use a digital signal to provide shaped pulses to a front end channel circuit (e.g., front end channel circuit 151). As illustrated, desired analog signal 505 may comprise a sinusoidal shape. However in other embodiments, desired analog signal 505 may comprise square waves, shaped square waves, triangle waves, any other type of shaped wave, and any combination thereof. In some embodiments, the frequency of light drive pulses 515 remains constant, while the widths of light drive pules 515 may be varied to match the magnitude of desired analog signal 505. For example, light drive pulses 515 that correspond in time to the high-amplitude portion of the analog signal 505 may have greater width than light drive pulses 515 that correspond in time to the low-amplitude portions of the desired analog signal 505.

FIG. 5B shows an illustrative plot of a detector signal that may be generated by a sensor (e.g., front end channel circuit 151 of FIG. 1) in accordance with some embodiments of the present disclosure. For example, FIG. 5B may illustrate an output signal generated by the front end channel circuit in response to light generated by a light source that is driven by light drive pulses 515. As shown in FIG. 5B, the response time of the front end channel circuit may be substantially longer than the period of the pulse frequency of light drive pulses 515. Consequently, front end channel signal 520 (e.g., detector current of the front end channel circuit 151) may increase during light drive pulses 515, and be attenuated somewhat (but not completely) during the periods between light drive pulses 515. In other words, the front end channel circuit may effectively integrate the signal received from the light source to generate front end channel signal 520 that generally matches desired analog signal 505. For purposes of illustration, FIG. 5B shows light drive pulses 515 having 22 pulses per one pulse of the desired analog signal 505). However, in some embodiments, light drive pulses 515 may have any number of pulses per one pulse of the desired signal. In some embodiments, the front end channel signal 520 may be a relatively rough saw-toothed shaped version of desired analog signal 505 when the light drive pulses 515 comprise a relatively low frequency. It will be understood that in some embodiments, light drive pulses 515 may have a significantly higher frequency resulting in the front end channel signal 520 that more closely approximates desired analog signal 505. For example, the desired analog signal 505 may comprise a sine wave repeated at 625 Hz, while the light drive pulses 515 may have a pulse frequency of 16 Mhz. In this example, one cycle of desired analog signal 505 may be represented by 25,600 (16000000/625) digital pulses of light drive pulses 515.

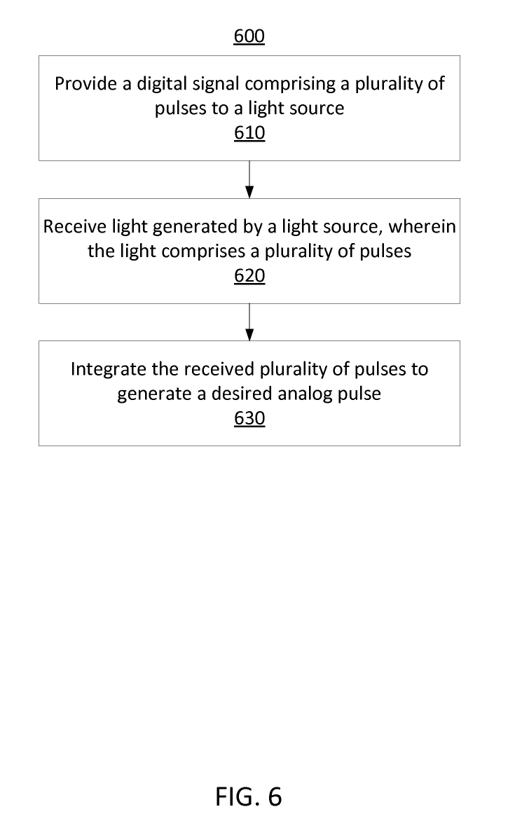

FIG. 6 shows an illustrative flow diagram 600 including steps for providing power to a light source in accordance with some embodiments of the present disclosure. In some embodiments, the steps of flow diagram 600 may be carried out by system 100 of FIG. 1. In some other embodiments, the steps of flow diagram 600 may be carried out by any other suitable PPG system or device.

Step 610 may include providing a digital signal comprising a plurality of pulses to a light source. In some embodiments, the light source may comprise one or more of LEDs or other light sources. In some embodiments, the digital signal may be provided by a light drive circuit (e.g., light drive circuit 120 of FIG. 1). In some embodiments, the plurality of pulses of the digital signal may comprise at least one pulse frequency and at least one corresponding period. In some embodiments, the plurality of pulses may correspond to digitally modulated pulses representative of a desired analog signal. For example, the plurality of pulses may comprise PWM square waves, PAM square waves, PDM square waves, PPM or any combination thereof. The plurality of pulses may comprise at least one feature that may be adjusted to vary power provided to the light source. The plurality of pulses may comprise at least one feature that may be adjusted to vary relative phase of the digital signal provided to the light source, as described in more detail below.

In some embodiments, where the plurality of pulses comprise a PWM signal, the width of the pulses may be adjustable while the frequency of the pulses may remain constant. In some embodiments, the at least one adjustable feature of the plurality of pulses may comprise at least one of pulse frequency, pulse density, pulse amplitude, pulse positions or pulse code or any combination thereof. In some embodiments, the power may be provided to the light source only during the high states of the digital signal. In some embodiments, the power may be provided at fixed voltage (or fixed current). In some embodiments, no power (or reduced power) may be provided to the light source during the period between pulses.

Step 620 may include receiving light generated by a light source and attenuated by body tissue of a patient. The light may arrive in the form of a plurality of pulses. In some embodiments, the received light may comprise a received plurality of pulses which were generated by the light source in response to receiving the digital signal. The received plurality of pulses may comprise at least one pulse frequency and at least one associated period. In some embodiments, the light may be received by a detector (e.g., detector 140 of FIG. 1) of the front end channel. In some embodiments, the detector may receive the light that comprises a plurality of pulses and, in response, transmit the plurality of received pulses to a processing circuit (e.g., front end processing circuit 150 of FIG. 1) of the front end channel. For example, the detector may generate an electrical current representative of the received plurality of pulses, while the processing circuit may be configured to receive and process the generated electrical current.

Step 630 may include integrating the received plurality of pulses to generate a desired analog pulse. In some embodiments, the received plurality of pulses may be integrated by the front end channel. In some embodiments, the front end channel may comprise a response time. The response time of the front end channel may be longer than the at least one pulse period of the received plurality of pulses. In some embodiments, the front end channel may not have time to settle after receiving a first pulse but before receiving a second pulse of the plurality of received pulses. Consequently the output signal of the front end channel may increase during each of the plurality of pulses, and be attenuated somewhat (but not completely) during the periods between each pulse. Consequently, the output of the front end channel may be an integrated version of the received plurality of pulses.

In some embodiments, the front end processing circuit may be configured to determine a phase shift present in the arrival time of the centroid of the received plurality of pulses. The phase shift may occur due to alignment of the plurality of pulses. For example, left-aligning pulses with variable width may result in a right shift of the phase. The determined phase shift may be used to re-optimize the digital signal in order to improve detectability of physiological data inferred from the plurality of received pulses. In some embodiments, the front end channel may be configured to provide an indication of phase delay based on a response time of each individual frequency component of the plurality of received pulses. The determined phase delay may be used to modify the digital signal sent to the light source. For example, the digital signal may be modified such, that the back end processing circuit (e.g., back end processing circuit of FIG. 1) receives the integrated output of the front end channel in an expected phase or at an expected time. In some embodiments, the response time of each individual frequency component of the plurality of received pulses may be used to modify the modulation of the digital signal. In some embodiments, the modulation of the digital signal may be modified to optimize the plurality of pulses for detectability of physiologic parameters or for Vernier differential adjustments. In some embodiments, the back end processing circuit may be configured to compensate or correct the integrated output of the front end channel.

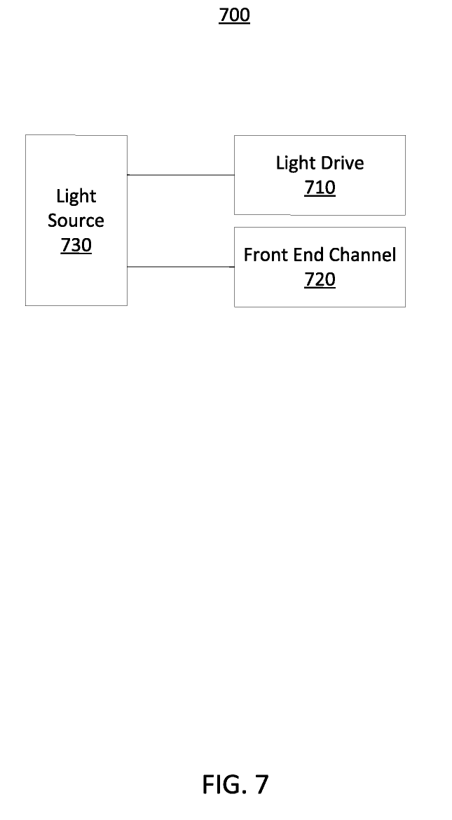

FIG. 7 shows a block diagram of an illustrative system 700 for light drive signal generation in accordance with some embodiments of the present disclosure. System 700 may include light drive 710, light source 730, and front end channel 720. The light drive 710 may include, for example, one or more elements of light drive circuit 120 of FIG. 1. The light source 730 may include, for example, one or more elements of light source 130 of FIG. 1. Front end channel 720 may include, for example, one or more elements of front end channel circuit 151 and detector 140 of FIG. 1. In other embodiments, each of the light drive 710, light source 730, and front end channel 720 may comprise any type of hardware or any type of combination of hardware and software necessary to perform the functions described below. For example, each of the light drive 710, light source 730, and front end channel 720 may comprise a processor and a memory. The memories may include any suitable transitory and/or non-transitory computer-readable media capable of storing information that can be interpreted by the processors. Processors (e.g., processor 172) may be adapted to execute software stored in the memories as a part of performing the functions described herein. In some embodiments, light drive 710, light source 730, and front end channel 720 may include hardware components (e.g., processor, controller) or a combination of hardware and software components. In some embodiments, the system 700 may be configured to perform steps 610, 620, and 630 of FIG. 6.

The light drive 710 may be configured to provide a digital signal comprising a plurality of pulses to a light source 730. The digital signal may include a plurality of pulses. In some embodiments, the plurality of pulses may comprise at least one pulse frequency and at least one corresponding period. In some embodiments, the plurality of pulses may correspond to digitally modulated pulses representative of a desired analog signal. For example, the plurality of pulses may comprise PWM square waves, PAM square waves, PDM square waves, PPM or any combination thereof. The light drive 710 may be configured to vary power provided to the light source 730 according to at least one adjustable feature of the plurality of pulses.

In some embodiments, where the plurality of pulses comprise a PWM signal, the width of the pulses may be adjustable while the frequency of the pulses may remain constant. In some embodiments, the at least one adjustable feature of the plurality of pulses may comprise at least one of frequency of the pulses, pulse density of the pulses, pulse amplitude of the pulses, pulse positions of the pulses or pulse code of the pulses or any combination thereof. In some embodiments, the light drive 710 may be configured to provide power to the light sources only during the high states of the digital signal. In some embodiments, the power may be provided at fixed voltage (or fixed current). In some embodiments, the light drive 710 may be configured to provide no power (or reduced power) to the light source 730 during the period between pulses.

The front end channel 720 may be configured to receive a signal corresponding to the plurality of pulses generated by the light source 730. In some embodiments, the received signal may comprise light generated by the light source where the light comprises a received plurality of pulses. In some embodiments, the light may be received by the front end channel 720 after it was attenuated by body tissue of a patient. In some embodiments, the received signal may correspond to a plurality of pulses, generated by the light source 730 in response to receiving the digital signal. The received plurality of pulses may comprise at least one pulse frequency and at least one associated period. In some embodiments, the signal may be received by a detector of the front end channel 720. In some embodiments, a detector (not shown) may receive light that comprises a received plurality of pulses and transmit the plurality of received pulses to a processing circuit of the front end channel 720. For example, the detector may generate an electrical current representative of the received plurality of pulses, while the processing circuit of the front end channel 720 may be configured to receive and process the generated electrical current.

The front end channel 720 may further be configured to integrate the plurality of pulses of the received signal to create a desired analog pulse. In some embodiments, the front end channel 720 may comprise a response time. The response time of the front end channel 720 may be longer than the at least one period of the plurality of pulses of the received signal. In some embodiments, the front end channel 720 may not have time to settle after receiving a first pulse but before receiving a second pulse of the plurality of received pulses of the received signal. Consequently the output signal of the front end channel 720 may increase during the plurality of pulses, and be attenuated somewhat (but not completely) during the periods between the pulses. Consequently, the output of the front end channel 720 may be an integrated version of the received plurality of pulses of the received signal.

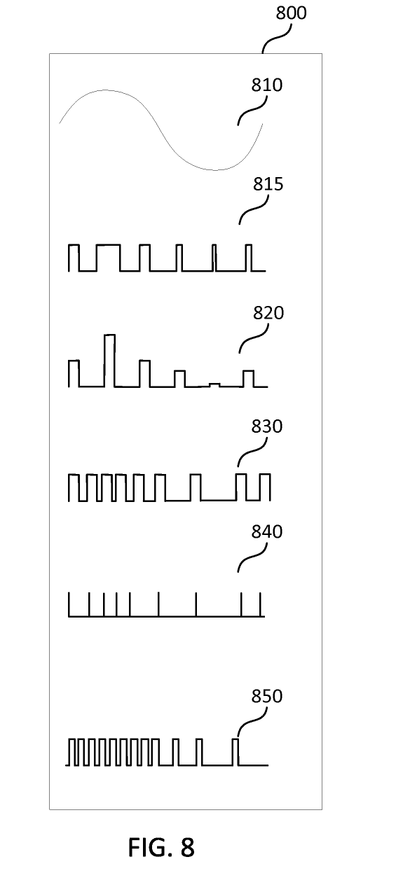

FIG. 8 shows a panel 800 of several illustrative plots of light drive signals that may be generated by a light drive system and provided to one or more light sources in accordance with some embodiments of the present disclosure. As discussed above, it may be desirable to drive one or more light sources such that a desired analog signal 810 is generated by a front end channel. In some embodiments, this is achieved by driving the light source with a plurality of square waves. For example, the light drive 710 of FIG. 7 may be used to drive the light source 730 with a plurality of square waves. The plurality of pulses may be a PWM signal 815 that represent the desired analog signal 810 (see, e.g., the above description of FIGS. 5A and 5B). In some embodiments, the plurality of pulses may be a Pulse Amplitude Modulated (PAM) signal 820. As shown in FIG. 8, the pulses of PAM signal 820 may have a constant carrier frequency but vary in amplitude. The PAM signal may cause the voltage provided to the light source to vary for each pulse. In some embodiments, the plurality of pulses may be a Pulse Density Modulated (PDM) signal 830. The pulses of PDM signal 830 may have a constant amplitude and carrier frequency, but have a varying pulse frequency, where more pulses are present during the times when the desired analog signal is in a high state. In some embodiments, the plurality of pulses may be a Pulse Position Modulated (PPM) signal 840. The pulses of PPM signal 840 may have a constant amplitude and a very short constant width, but have a varying delay between each pulse. In some embodiments, the plurality of pulses may be a Pulse Code Modulated (PCM) signal 850. PCM signal 850 may comprise pulses that digitally encode (using e.g., binary coding) a sample value of the desired analog signal. The received PCM signal may be subject to additional processing by either the detector or the front end channel. For example, the gain of the front end channel may be different depending on the position of the pulse. In all of these embodiments, the power provided to the one or more light sources varies in accordance with the variable feature of the modulated pulses.

In some embodiments, techniques may be used to enhance the resolution of the signal received by a front end channel (e.g., front end channel circuit 151 of FIG. 1) that receives light generated by a light source (e.g., light source 130 of FIG. 1) according to a digitally modulated light drive signal. For example, in some embodiments, a patient's tissue may return a lot of light, which may cause the system to decrease the power provided to the light source. This may be accomplished, for example, by only using shorter pulses in the PWM signal. This, in turn, may create a case where there are not enough discrete widths of pulses available to accurately represent the desired signal. In this case, other techniques, describes in detail below, may be used to enhance the resolution of the signal.

In one exemplary embodiment, the light drive signal may be PWM signal that has constant pulse frequency of 8 MHz. Additionally, in this example, it may be desired to transmit an analog signal to a single LED light source, where the desired analog signal is a sinusoidal pulse repeated at 625 Hz. Consequently, in this example, 12,800 (8000000/625) clock cycles are available to generate a complete cycle of the desired sine wave. An exemplary front end channel may be capable of sampling the output of the LED light source at a certain rate. In some embodiments, 40 points (samples) may be used to represent the sine wave. Consequently 320 (12,800/40) clock cycles can be used to modulate the width of each pulse of the plurality of pulses. In some embodiments, the light source may comprise both a red LED and an IR LED. If a first sine wave is transmitted over the red LED and a second sine wave is transmitted over the IR LED, 160 clocks may be used to vary the width of the pulses that drive the red LED and another 160 clocks may be used to vary the width of pulses that drive the IR LED. In some other embodiment, it may be desirable to have dark periods between powering on the red LED and the IR LED. In such embodiments, 80 (640/4) clocks may be used to vary the width of pulses that drive each of the IR and red LEDs, while another 160 clocks are dedicated to dark periods when no pulses are generated. It should be understood that aforementioned embodiment is provided as an example only, and in other embodiments, other suitable pulse frequencies, desired analog signals, numbers of samples, and other features may be used.

In the aforementioned exemplary embodiment, the width of the pulses may be modulated in the following manner: when the desired analog signal is at its lowest amplitude in the sine wave, an LED may be turned on for 0/320 clock cycles (0% pulse width) and when the desired analog signal is at the highest amplitude (peak) of the sine wave, the LED may be turned on for 320/320 clock cycles (100% pulse width). In some embodiments, turning on the LED on for 0/320 (0%) clock cycles or for 320/320(100%) clock cycles may be avoided, since such operations may change the LED pulse rate. Instead, a minimum of 3/320 (e.g., 1%) clock cycles and a maximum of 317/320 (99%) clock cycles pulse widths may be used.

In some embodiments, when the LED is run at full power, then 320 counts may be equivalent to an 8.3 bit DAC (i.e., Log₂(320)). This means that the sidelobes of the modulation frequency will be attenuated approximately 6.02 dB×8.3=50 dB from the intended frequency. However, if body tissue positioned between the light source and the front end channel returns a lot of light, it may be necessary to reduce the peak output of the LED from 100% to 10% of full scale. In some embodiments, the modulated pulses may be modulated up to a maximum width of only 32 clock cycles while clock cycles 33-320 may be always set to zero. In some other embodiments, any 288 of the 320 clocks cycles may be set to zero to achieve the same effect. This may result in the front end channel receiving a quantized sine wave similar to a 5 bit DAC and resulting in sidelobes being attenuated only 30 dB.

Additionally, several methods may be used to increase the resolution of a light drive signal provided to the light sources. In some embodiments, in addition to varying the pulse width of the pulses of the light drive signal, the pulse frequency may also be varied to exclude frequencies that alias to the region of the carrier frequency. For example, instead of using 320 clocks for each pulse, the number of clocks for each pulse may vary from 310-330 samples.

In some embodiments, the LED pulse frequency and width may be purposely varied in a way that spreads the noise from the pulse frequency across a wider frequency range and reduces the noise due to the pulses which would otherwise occur at a specific frequency. The spreading of the spectrum may be targeted such that none of the pulse rate energy aliases down to the region around the carrier frequency of the digital light drive signal.

In some embodiments, dithering of the digital light drive signal may be used to reduce quantization patterns. For example, a certain pulse width may be desired in a certain location of the digital light drive signal. Instead of generating a pulse with the desired width, two or more pulses with alternating widths are generated instead. The frequency of the two or more pulses may be set in such a way that the average width of these pulses is equal to the desired pulse width. Consequently, the desired pulse width is achieved, while quantization patterns are minimized. In some embodiments, dithering may be used with respect to any adjustable feature of the pulses of the digital light drive signal. For example, dithering may be applied to width, frequency, amplitude, density or any combination of these features.

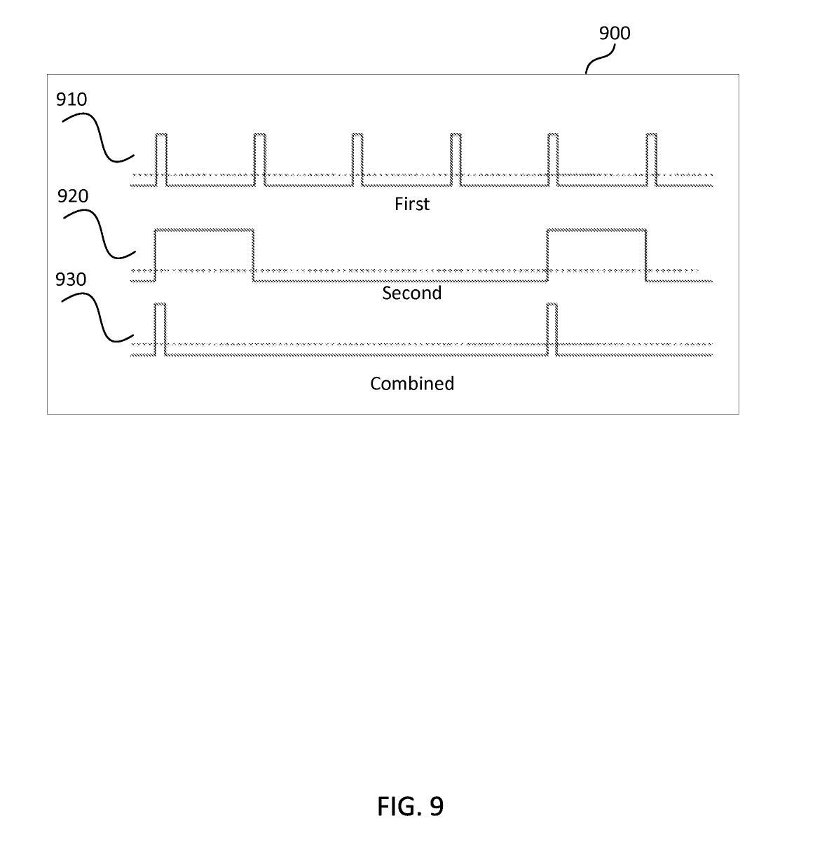

In some embodiments, pulse skipping may be used in order to modify the pulse frequency of the digital light drive signal in order to increase the resolution of the digital light drive signal. For example, the digital light drive signal may comprise a PWM signal. In this example, skipping some pulses of the light drive signal allows for additional frequency modulation of the PWM signal. FIG. 9 shows a panel 900 of illustrative timing diagrams for generating an exemplary light drive signal in accordance with some embodiments of the present disclosure. In particular, FIG. 9 shows and exemplary method of applying an exemplary pulse skipping technique to a PWM light drive signal 910. However, it should be understood that pulse skipping may be applied to any other kind of digitally modulated signal. In the shown embodiment, a second PWM modulated signal 920 is utilized for pulse skipping. In some embodiment, the pulses of the second PWM signal 920 may have a period that is a multiple of the period of the first PWM light drive signal 910. In some embodiments, pulse skipping may be accomplished by executing a logical AND command between the light drive signal 910 and the second PWM modulated signal 920 resulting in a combined signal 930 where some pulses are skipped. In other embodiments, pulse skipping may be accomplished by any other technique, such as storing multiple signals with skipped pulses and using them when they are needed.

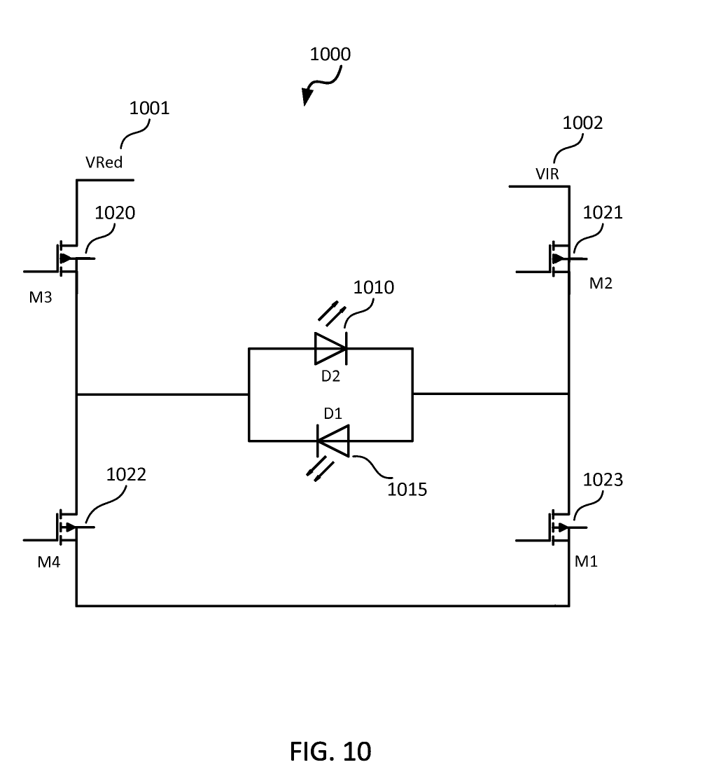

FIG. 10 is a circuit diagram of an exemplary circuit 1000 that may be used to provide light drive signals to a light source in accordance with some embodiments of this disclosure. Circuit 1000 may comprise a light source (e.g., light source 130 of FIG. 1) and a light drive (e.g., light drive 120 of FIG. 1). In some embodiments, circuit 1000 may have four transistors 1020, 1021, 1022, and 1023 in an H-bridge configuration configured to apply voltage in either direction across two LEDs 1010 and 1015 that are connected in an anti-parallel configuration (also referred to as a back-to-back configuration). In some embodiments, LED 1010 is a red LED, while LED 1015 is an IR LED. The Vred voltage source 1001 and VIR voltage source 1002 may comprise fixed voltage (or fixed current) power sources (e.g., the voltage may be fixed at 1.8V). The transistors 1020, 1021, 1022, and 1023 may be controlled by a processor (not shown) in order to selectively generate current across either of the two LEDs 1010 and 1015. For example, when the transistors 1020 and 1023 are enabled, LED 1010 will conduct current, while when the transistors 1021 and 1022 are enabled, LED 1015 will conduct current. The transistors 1020, 1021, 1022, and 1023 may be switched on and off at a very high frequency to provide a digitally modulated light drive signal to LEDs 1010 and 1015.

When the Vred and VIR voltage sources 1001 and 1002 are driven with constant voltage using a digitally modulated digital signal (e.g., a PWM signal), as described above, the circuit 1000 may have very high efficiency. Instead of regulating the LEDs 1010 and 1015 to a specified current, the LEDs 1010 and 1015 may be configured to produce as much current as physically possible given the specific fixed voltage sources (e.g., voltage sources 1001 and 1002) during each pulse of the digitally modulated signal. When the voltage sources are connected to LEDs 1010 and 1015 according to a digitally modulated light drive signal (e.g., a PWM signal), as described above, the LEDs 1010 and 1015 may produce a controlled/quantified amount of light (e.g., an approximated version of a desired analog signal). Circuit 1000 demonstrates an efficient way of driving LEDs 1001 and 1002 because it has nearly zero resistive losses. The only significant losses come from the switching losses of the transistors 1020, 1021, 1022, and 1023. The period between the on and off states of the transistors 1020, 1021, 1022, and 1023 is the only major loss in the circuit 1000.

It will be understood that the configuration of FIG. 10 is merely illustrative and various modifications can be made to the configuration of circuit 1000 within the scope of this disclosure. For example, while VRed and VIR voltage sources 1001 and 1002 are shown as being separate, a common voltage source may be used in some embodiments. As another example, circuit 1000 may be modified to include additional lights sources and additional transistors for turning on and off each of the light sources.

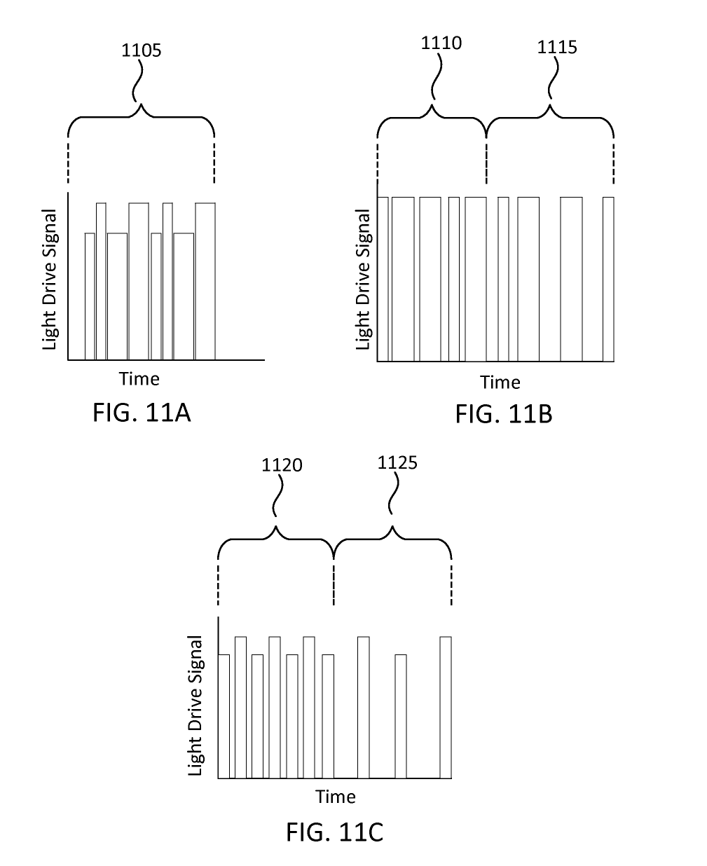

FIGS. 11A-C shows several illustrative plots of light drive signals that may be provided by a light drive in accordance with some embodiments of the present disclosure. The light drive signals may be provided by a light drive (e.g., light drive circuit 120) to a light source (e.g., light source 130) of a PPG system (e.g., system 100). The light drive signals may comprise a plurality of pulses and power may be provided to the light sources during each pulse. Each of the light drive signals may comprise at least two adjustable features. In some embodiments, the adjustable features may be used to vary the power applied to the light source. In some embodiments, no power (or reduced power) is provided to the light source during the periods between each of the plurality of pulses. The light generated by the light source may, in-turn, be received by a front end channel circuit (e.g., front end channel circuit 151).

In some embodiments, the plurality of pulses may comprise at least one pulse frequency and at least one period associated with the at least one pulse frequency. The front end channel circuit may also comprise a response time that may be longer than the at least one period associated with the at least one pulse frequency. In some embodiments, the front end channel circuit may be configured to integrate the received light to create a desired analog signal. For example, the received light may be integrated due to the front end channel circuit not having enough time to settle between receiving the light generated by the light sources during any two adjacent pulses of the plurality of pulses.

In some embodiments, the two adjustable features of the plurality of pulses may comprise two of the following: the width of each pulse of the plurality of pulses, the amplitude of each pulse of the plurality of pulses, and the pulse frequency of each pulse of the plurality of pulses. For example, in some embodiments the two adjustable features of the plurality of pulses may be the width of the plurality of pulses and the amplitude of the plurality of pulses. In some embodiments, the two adjustable features of the plurality of pulse may be the width of the plurality of pulses and the pulse frequency of the plurality of pulses. In some embodiments, the two adjustable features of the plurality of pulses may be the amplitude of the plurality of pulses and the pulse frequency of the plurality of pulses. The light drive signals that comprise two adjustable features may result in a greater number of distinct signal power levels that may be generated. For example, if the light drive signal comprises pulse width modulated pulses that are further amplitude modulated, a greater number of distinct signal power levels can be provided.

FIG. 11A shows an exemplary plot of some embodiments of the disclosure where the light drive signal comprises a digital signal 1105 that comprises a plurality of pulses. In some embodiments, the digital signal 1105 comprises two adjustable features (i.e., features that comprise two or more discrete states): width of the pulses and amplitude of the pulses. In the shown embodiment, the digital signal 1105 comprises pulses that have two different possible widths and two different possible amplitudes, however, any number of possible widths and possible amplitudes may be used. In some embodiments, the light drive circuit may provide power to the light source only during each pulse of the digital signal 1105. If the pulse width were the only adjustable feature of the plurality of pulses, the number of distinct signal power levels that may be generated would be limited to the number of possible pulse widths. However, by providing a variable voltage at two or more discrete levels to the light source, voltage may be adjusted according to the amplitude of each pulse of the plurality of pulses. Accordingly, by using adjustable pulse width and amplitude, a higher number of distinct signal power levels may be generated compared to embodiments where the light drive signal comprises only a single adjustable feature.

FIG. 11B shows an exemplary plot of some embodiments of the disclosure where the light drive signal comprises a digital signal that comprises a plurality of pulses 1110 and 1115. In some embodiments, the plurality of pulses 1110 and 1115 comprise two adjustable features: width of the pulses and frequency of the pulses. In some embodiment, the plurality of pulses 1110 and 1115 comprises pulses that have two different possible widths and two different possible pulse frequencies, however, any number of possible widths and possible pulse frequencies may be used. In some embodiments, the light drive circuit may provide power to the light sources only during each pulse of the plurality of pulses 1110 and 115. If the pulse width was the only adjustable feature of the plurality of pulses, the number of different signals that may be generated would be limited to the number of possible pulse widths. However, in some embodiments the light drive may be configured to provide pulses 1110 at one frequency and pulses 1115 at a different frequency. In some embodiments, the change in pulse frequency may vary the time between the pulses of the plurality of pulses 1110 and 1115. In some embodiments, the change in pulse frequency may also increase or decrease the number of possible widths of the pulses. For example, lower pulse frequency such as those of pulses 1115 may provide a longer time period during which a pulse may be provided, consequently such a pulse may comprise more possible widths. In some embodiments, such a pulse may comprise the width that is equal to the entire time period allowed by the pulse frequency for proving a pulse. Accordingly, by using adjustable width and frequency, a higher number of distinct signal power levels may be generated compared to embodiments where the light drive signal only comprises a single adjustable feature.

FIG. 11C shows and exemplary plot of some embodiments of the disclosure where the light drive signal comprise a digital signal that comprise a plurality of pulses 1120 and 1125. In some embodiments, the plurality of pulses 1120 and 1125 comprise two adjustable features: amplitude of the pulses and frequency of the pulses. In the shown embodiment, the plurality of pulses 1120 and 1125 comprises pulses that have two different possible amplitudes and two different possible pulse frequencies, however, any number of possible amplitudes and possible pulse frequencies may be used. In some embodiments, the light drive circuit may provide power to the light sources only during each pulse of the plurality of pulses 1105. If the pulse amplitude was the only adjustable feature of the plurality of pulses, the number of distinct signal power levels that may be generated would be limited to the number of possible pulse amplitudes. However, in some embodiments the light drive may be configured to provide pulses 1120 at one frequency and pulses 1125 at a different frequency. In some embodiments, the change in pulse frequency may vary the time between pulses of the plurality of pulses 1120 and 1125 creating more possible distinct signal power levels. Accordingly, by using adjustable pulse amplitude and frequency a higher number of distinct signal power levels may be generated compared to embodiments where the light drive signal only comprises a single adjustable feature.

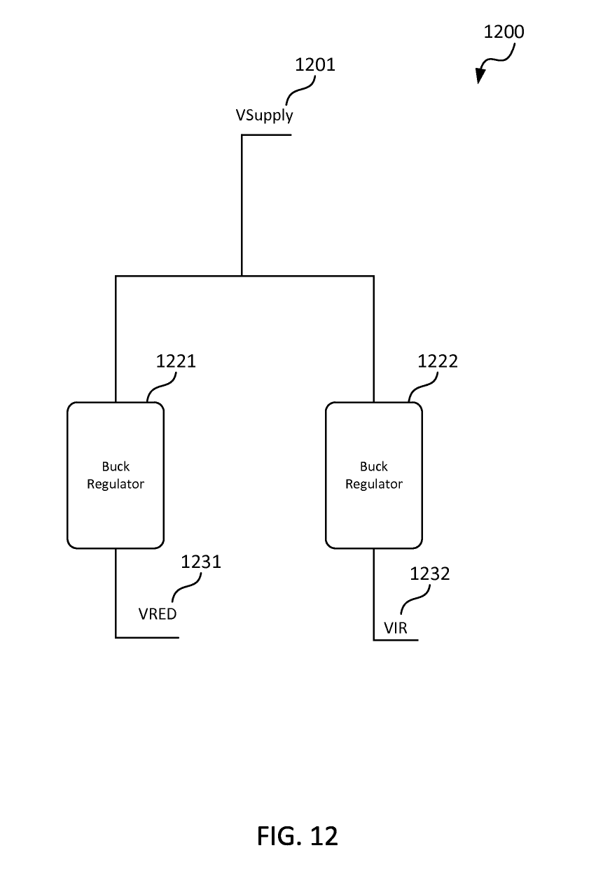

FIG. 12 shows a circuit diagram of an illustrative system for adjusting voltage of a power source in accordance with some embodiments of the present disclosure. In particular, FIG. 12 shows a circuit 1200 that is capable of adjusting voltage of a power source of a PPG system (e.g., system 100 of FIG. 1). In particular, circuit 1200 allows a PPG system to generate light drive signals that comprise pulses with variable amplitude. In some embodiments, the circuit 1200 may be connected to circuit 1000 of FIG. 10, and may be used to provide variable voltage to LEDs 1010 and 1015. In the shown embodiment, the circuit 1200 regulates voltage regulation by using buck regulators 1221 and 1222. Buck regulators are extremely efficient at stepping down voltage by using on or more inductors to store and/or provide energy at appropriate times. However, in some other embodiments, different ways of regulating power may be used, e.g. it possible to regulate power by using a linear regulator, a boost regulator, a buck/boost regulator, a charge pump, a low-dropout regulator (LDO) or other types of circuits or devices suitable for this purpose.

In some embodiments, light sources of a PPG system (e.g., LEDs 1010 and 1015 of FIG. 10) may be driven over a greater dynamic range by controlling the current and/or voltage amplitude applied to the light sources. In some embodiments, voltage regulators (such as buck regulators 1221 and 1222) may be used to control the current and/or voltage amplitude. The use of buck regulators 1221 and 1222 may result in voltages Vred 1231 and VIR 1232 being smaller than the supply voltage 1201. For example, each of the buck regulators 1221 and 1222 may be configured to step down voltage provided by the Vsupply 1201 to desired voltage. In some embodiment, this may result in lowering the voltage amplitude applies to the light sources. For example, if circuit 1200 is connected to circuit 1000 of FIG. 10, the voltage provided to the LED 1010 may be controlled by buck regulator 1221, while the voltage provided to the LED 1015 may be controlled by buck regulator 1222. The ability to control the current and/or voltage amplitude in addition to the ability to control pulse width allows the PPG system to generate more possible distinct signal power levels. In other embodiment, other methods or techniques of controlling the amplitude of Vred and VIR voltages 1231 and 1232 may be used.

It will be understood that the aforementioned techniques are not limited to PPG systems, and may be applied to any suitable signal processing in any suitable system. For example, the techniques may be applied to electrical signals additionally or alternatively to applying it to optical signals. In another example, the techniques may be applied additionally or alternatively to respiration signals.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

1. A photoplethysmography (PPG) system, the system comprising:
a light drive circuit configured to provide a digital signal that provides power to a light source, the digital signal comprising a plurality of pulses, wherein at least one feature of the plurality of pulses is adjustable to vary the power provided to the light source, the light drive circuit being further configured to provide power to the light source during each pulse of the plurality of pulses, and wherein the plurality of pulses comprises one or more periods;
a front end channel circuit configured to receive light generated by the light source in response to the plurality of pulses after the light has been attenuated by a body tissue, wherein the front end channel circuit comprises a response time; and
wherein the one or more periods of the plurality of pulses are substantially shorter than the response time of the front end channel circuit.

2. The system of claim 1, wherein the at least one adjustable feature of the plurality of pulses comprises at least one of a width of the plurality of pulses, a frequency of the plurality of pulses, a density of the plurality of pulses, an amplitude of the plurality of pulses, a position of the plurality of pulses, and pulse code of the plurality of pulses.

3. The system of claim 1, wherein the light drive circuit is further configured to provide no power or reduced of power to the light source during intervals between the plurality of pulses.

4. The system of claim 1, wherein the power provided by the light drive circuit comprises at least one of fixed voltage and fixed current during each pulse of the plurality of pulses.

5. The system of claim 1, wherein the front end channel circuit is further configured to integrate the received light to generate a desired analog pulse.

6. The system of claim 5, wherein the desired analog pulse comprises at least one of a sinusoidal wave and a shaped square wave.

7. The system of claim 1, wherein the light drive signal comprises pulses that are dithered with respect to the at least one adjustable feature.

8. The system of claim 1, wherein the light source comprises two LEDs in a back-to-back configuration.

9. The system of claim 8, wherein the light drive circuit comprises a plurality of transistors in an H-bridge configuration, such that the light drive circuit is capable of separately providing power to each of the two LEDs.

10. The system of claim 1, wherein the light drive circuit comprises at least one of a buck regulator, a linear regulator, a boost regulator, a buck/boost regulator, a charge pump, and a low-dropout regulator for varying the power provided to the light source.

11. The system of claim 1, wherein a pulse skipping template is applied to the digital signal.

12. A photoplethysmography (PPG) system, the system comprising:
a light drive configured to provide a digital signal that provides power to a light source, the digital signal comprising a plurality of pulses, the light drive being further configured to provide power to the light source during each pulse of the plurality of pulses; and
a front end channel configured to receive pulses of light generated by the light source in response to the plurality of pulses of the digital signal, the pulses of light being attenuated by a body tissue, and wherein the front end channel is further configured to integrate the pulses of light to generate a desired analog pulse.

13. The system of claim 12 wherein the plurality of pulses of the digital signal comprises at least one adjustable feature to vary the power provided to the light source, and wherein the pulses of light comprise one or more periods, and wherein the front end channel comprises a response time, and wherein the one or more periods of the pulses of light are substantially shorter than the response time of the front end channel.

14. The system of claim 13, wherein the adjustable feature of the plurality of pulses of the digital signal comprises at least one of a width of the plurality of digital pulses, a frequency of the plurality of digital pulses, a density of the plurality of digital pulses, an amplitude of the plurality of digital pulses, a position of the plurality of digital pulses, and a pulse code of the plurality of digital pulses.

15. A method for providing power to a light source of a photoplethysmography (PPG) device, the method comprising:
providing power to the light source according to a digital signal, the digital signal comprising a plurality of pulses, wherein at least one feature of the plurality of pulses is adjustable to vary the power provided to the light source, wherein the power is provided to the light source during each pulse of the plurality of pulses, and wherein the plurality of pulses comprises one or more periods;
receiving, by a front end channel of the PPG device, light generated by the light source in response to the plurality of pulses after the light has been attenuated by a body tissue, wherein the front end channel comprises a response time, and wherein the one or more periods of the plurality of pulses are substantially shorter than the response time of the front end channel.

16. The method of claim 15, wherein the at least one adjustable feature of the plurality of pulses comprises at least one of a width of the plurality of pulses, a frequency of the plurality of pulses, a density of the plurality of pulses, an amplitude of the plurality of pulses, a position of the plurality of pulses, and a pulse code of the plurality of pulses.

17. The method of claim 15, further comprising integrating the light received by the front end channel to generate a desired analog pulse.

18. A method for providing power to a light source of a photoplethysmography (PPG) device, the method comprising:
providing a digital signal that provides power to the light source, the signal comprising a plurality of pulses, wherein the power is provided to the light source during each pulse of the plurality of pulses;
receiving, by a front end channel of the PPG device, pulses of light generated by the light source in response to the plurality of pulses of the digital signal, the pulses of light being attenuated by a body tissue; and
integrating the pulses of light to generate a desired analog pulse.

19. The method of claim 18 wherein the plurality of pulses of the digital signal comprises at least one adjustable feature to vary the power provided to the light source, wherein the pulses of light comprise one or more periods, and wherein the front end channel comprises a response time, and wherein the one or more periods of the pulses of light are substantially shorter than the response time of the front end channel.

20. The method of claim 19, wherein the adjustable feature of the plurality of pulses of the digital signal comprises at least one of a width of the plurality of digital pulses, a frequency of the plurality of digital pulses, a density of the plurality of digital pulses, an amplitude of the plurality of digital pulses, a position of the plurality of digital pulses and a pulse code of the plurality of digital pulses.

21-40. (canceled)
